(19) United States
(12) Patent Application Publication
(10) Pub. No.: US 2024/0099835 A1
(43) Pub. Date: Mar. 28, 2024

(54) LEAFLETS AND LEAFLET SEPARATORS FOR PROSTHETIC VALVES

(71) Applicant: EDWARDS LIFESCIENCES CORPORATION, Irvine, CA (US)

(72) Inventors: Tamir S. Levi, Zikhron Yaakov (IL); Anatoly Dvorsky, Haifa (IL)

(21) Appl. No.: 18/530,682

(22) Filed: Dec. 6, 2023

Related U.S. Application Data
(63) Continuation of application No. PCT/US2022/032303, filed on Jun. 6, 2022.
(60) Provisional application No. 63/324,795, filed on Mar. 29, 2022.
(60) Provisional application No. 63/279,504, filed on Nov. 15, 2021.
(60) Provisional application No. 63/197,559, filed on Jun. 7, 2021.

(51) Int. Cl. A61F 2/24

(52) U.S. Cl. CPC A61F 2/2418; A61F 2210/0057; A61F 2220/0075; A61F 2230/0076

(57) ABSTRACT

A prosthetic valve, constituted of: a frame movable between a radially compressed and a radially expanded state; and a plurality of leaflets secured to the frame.

### CROSS REFERENCE TO RELATED APPLICATIONS

This application is a continuation of International Application No. PCT/US2022/032303, filed Jun. 6, 2022, which claims benefit of U.S. Provisional Application No. 63/197,559, filed on Jun. 7, 2021, U.S. Provisional Application No. 63/279,504 filed on Nov. 15, 2021, and U.S. Provisional Application No. 63/324,795, filed on Mar. 29, 2022, the contents of each of which are herein incorporated by reference in their entirety.

### FIELD OF THE INVENTION

The invention relates generally to the field of prosthetic valves.

### BACKGROUND OF THE INVENTION

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic valve over a catheter and implanting it over the native malfunctioning valve, have been developed over the years.

Different types of prosthetic valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

A prosthetic valve conventionally includes a circumferential frame that can be a metallic frame configured to transition between compressed and expanded states, and soft components sutured thereto, such as a leaflet assembly composed of a plurality of leaflets attached to the frame via a plurality of commissure assemblies, and configured to regulate blood flow through the prosthetic valve, as well as a skirt that can prevent perivalvular leakage as further serve as an intermediate means of attachment of the leaflet assembly, along a lower scalloped edge thereof, to the frame. The average human heart beats about 100,000 times per day. Thus, such soft components may be subject to wear due to ongoing movement thereof relative to the frame, as the leaflets move between open and closed positions during systolic and diastolic phases of the blood flow through the valve.

The leaflets can include tabs at both ends, such that two tabs of each two adjacent leaflets are secured to each other to form a commissure assembly by which they are coupled to the frame. Conventional commissure assemblies are formed such that the portions of the leaflets in the vicinity of the frame, e.g. in or in close proximity to the commissure assemblies, are pressed against each other both in their open and closed positions. This direct contact of these portions of the leaflets can promote tissue overgrowth or pannus formation while the prosthetic valve is implanted within a patient's body, which in turn will result in a relatively rigid region of the leaflets that extends radially inward over time, and may limit the effective orifice area (EOA) of the valve in the open position of the leaflets.

Another drawback of some prosthetic valves is that conventional leaflets are typically designed as flattenable components that assume a somewhat folded configuration when attached to the frame, but can be unbent and flattened when placed on a flat plane, for example prior to prosthetic valve assembly. It has been observed that lateral folds may be formed across the leaflets in their open position, which can result in flow separation due to excessive momentum loss immediately after flowing past the fold. This disrupts the flow with turbulence and flow eddies, which cause the free ends of the leaflets to flutter. The stresses of such flutter can lead to fatigue failure over time. Another drawback associated with utilization of flattenable leaflets relates to their limited robustness to geometrical changes of the frame, as their design and dimensions are adapted to provide adequate EOA in their open position and proper sealing by coapting against each other in their closed position, in a relatively limited geometry and size of the prosthetic valve.

### SUMMARY

Accordingly, it is a principal object of the present invention to overcome at least some of the disadvantages of prior art prosthetic valves.

Additional features and advantages of the invention will become apparent from the following drawings and description.

In a representative example, a prosthetic valve can include a frame having an inflow end and an outflow end, and a plurality of leaflets secured to the frame. The frame is movable between a radially compressed and a radially expanded state. Each leaflet can include a leaflet first face and a leaflet second face opposing the leaflet first face, a free edge defining a free edge midpoint, and an opposing cusp edge. The cusp edge defines a cusp edge midpoint and first and second leaflet body sidelines, each leaflet body sideline extending from the cusp edge midpoint to the free edge. Each of the plurality of leaflets is not flattenable.

In another representative example, a prosthetic valve can include a frame having an inflow end and an outflow end, and a plurality of leaflets secured to the frame. The frame is movable between a radially compressed and a radially expanded state. Each leaflet can include a leaflet first face and a leaflet second face opposing the leaflet first face, a free edge defining a free edge midpoint, and an opposing cusp edge. The cusp edge defines a cusp edge midpoint and first and second leaflet body sidelines, each leaflet body sideline extending from the cusp edge midpoint to the free edge. The plurality of leaflets are secured to each other at a plurality of commissure assemblies, each of the plurality of commissure assemblies extending from a commissure assembly outflow end to a commissure assembly inflow end. The plurality of commissure assemblies are secured to the frame. A center of coaptation of the plurality of leaflets is located between the commissure assembly inflow ends and the inflow end of the frame.

In another representative example, a prosthetic valve can include a frame having an inflow end and an outflow end, and a plurality of leaflets secured to the frame. The frame is movable between a radially compressed and a radially expanded state. Each leaflet can include a leaflet first face and a leaflet second face opposing the leaflet first face, a free edge defining a free edge midpoint, and an opposing cusp edge. The cusp edge defines a cusp edge midpoint and first and second leaflet body sidelines, each leaflet body sideline extending from the cusp edge midpoint to the free edge. For each of the plurality of leaflets, the respective leaflet is curved from the first leaflet body sideline to the second leaflet body sideline.

In another representative example, a prosthetic valve can include a frame movable between a radially compressed and a radially expanded state, and a plurality of leaflets. The frame can include a plurality of commissure windows. Each commissure window can include two sidewalls and define a commissure window inner surface and a commissure window outer surface. Each leaflet can include two opposite tabs, wherein the tabs of adjacent leaflets are coupled to each other to form commissure assemblies. Each commissure assembly can be coupled to a corresponding commissure window. Each commissure assembly can include a separator having a separator thickness and defining a separator inner end, wherein the separator is disposed between portions of the tabs extending through the commissure window.

In another representative example, a prosthetic valve can include a frame having an inflow end and an outflow end, and a plurality of leaflets secured to the frame. The frame is movable between a radially compressed and a radially expanded state. Each leaflet has a leaflet first face and a leaflet second face opposing the leaflet first face, a free edge and an opposing cusp edge. The cusp edge defines first and second leaflet body sidelines, each leaflet body sideline extending from a midpoint of the cusp edge midpoint to the free edge. Each of the plurality of leaflets can include a plurality of lateral folds extending between the leaflet body sidelines.

Unless otherwise defined, all technical and scientific terms used herein have the same meaning as commonly understood by one of ordinary skill in the art to which this invention pertains. In case of conflict, the patent specification, including definitions, governs. As used herein, the articles “a” and “an” mean “at least one” or “one or more” unless the context clearly dictates otherwise. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF DRAWINGS

For a better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred examples of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice. In the accompanying drawings:

FIGS. 1A-1E illustrate various high-level views of a prior art prosthetic valve;

FIGS. 2A-2E illustrate various high-level views and features of a leaflet for a prosthetic valve, in accordance with some examples;

FIG. 3A illustrates a high-level view of a prosthetic valve comprising a plurality of leaflets of FIGS. 2A-2E, in an almost closed position, in accordance with some examples;

FIGS. 3B-3F illustrate various high-level views of the leaflets of FIG. 3A in different positions, in accordance with some examples;

FIGS. 3G-3H illustrate various high-level views of the prosthetic valve of FIG. 3A, with the plurality of leaflets in an open position, in accordance with some examples;

FIGS. 4A-4B illustrate various views of a relationship between the leaflets of FIGS. 2A-3F and the frame of FIGS. 3A and 3G;

FIGS. 5A-5C illustrate various high-level views of a prosthetic valve, with the center of coaptation in a lowered position, in accordance with some examples;

FIGS. 6A-6B illustrate various high-level views of a leaflet for a prosthetic valve, where a portion of the leaflet is below a plane defined by the end thereof, in accordance with some examples;

FIGS. 7A-7B illustrate various high-level partial views of a commissure assembly with a separator, in accordance with some examples.

FIGS. 8A-8B illustrate the commissure assembly of FIGS. 7A-7B with various radial lengths of the separator, in accordance with some examples.

FIGS. 9A-9C illustrate the commissure assembly of FIGS. 7A-7B with various positions of an inner end of the separator, in accordance with some examples.

FIGS. 10A-10B illustrate various high-level partial views of another configuration of a commissure assembly with a separator, in accordance with some examples.

FIGS. 11A-11B illustrate various high-level partial views of a commissure assembly with a separator formed as part of a coupling member, in accordance with some examples.

FIGS. 12A-12B illustrate high-level partial perspective views of a commissure assembly with various types of T-shaped separators, in accordance with some examples.

FIG. 13 illustrates a high-level perspective view of a prosthetic valve, in accordance with some examples.

FIG. 14 illustrates a high-level perspective view of a leaflet with lateral folds, in accordance with some examples.

FIGS. 15A-15B illustrate steps of forming leaflets with lateral folds by forging, in accordance with some examples.

FIG. 16 illustrates a high-level perspective view of another type of a prosthetic valve, in accordance with some examples.

FIGS. 17A-17B illustrate a two-layered leaflet with lateral folds in a free and stretched state thereof, respectively, in accordance with some examples.

FIG. 18 illustrates an exemplary delivery apparatus carrying a balloon expandable prosthetic valve.

### DETAILED DESCRIPTION OF CERTAIN EXAMPLES

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein. In order to avoid undue clutter from having too many reference numbers and lead lines on a particular drawing, some components will be introduced via one or more drawings and not explicitly identified in every subsequent drawing that contains that component.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right“, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. Additionally, the terms “has”, “exhibits” or “includes” means “comprises”.

As used herein, the terms “integrally formed” and “unitary construction” refer to a construction that does not include any welds, fasteners, or other means for securing separately formed pieces of material to each other.

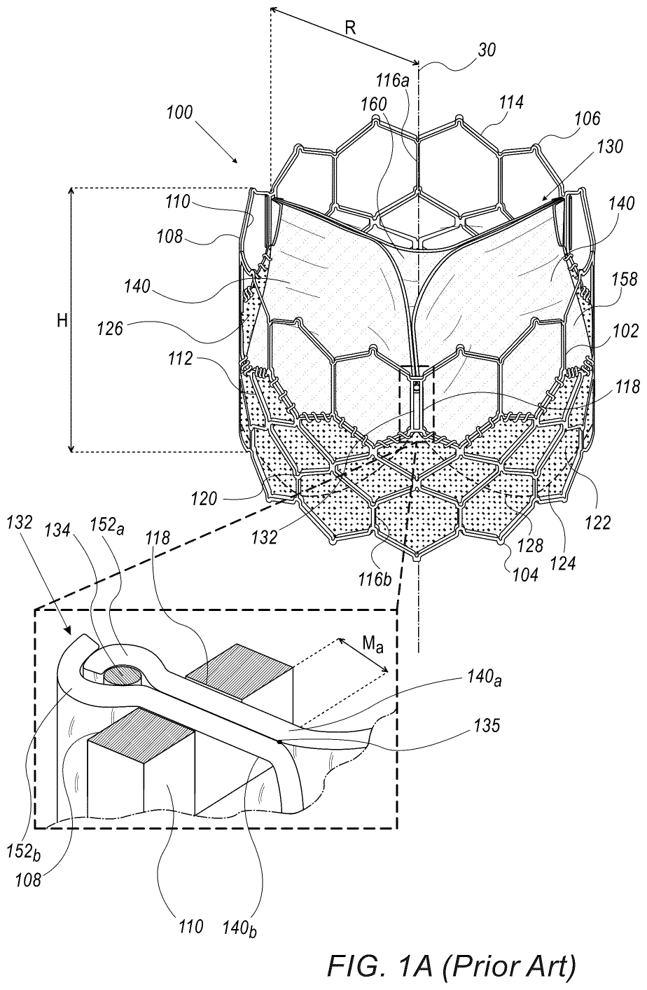

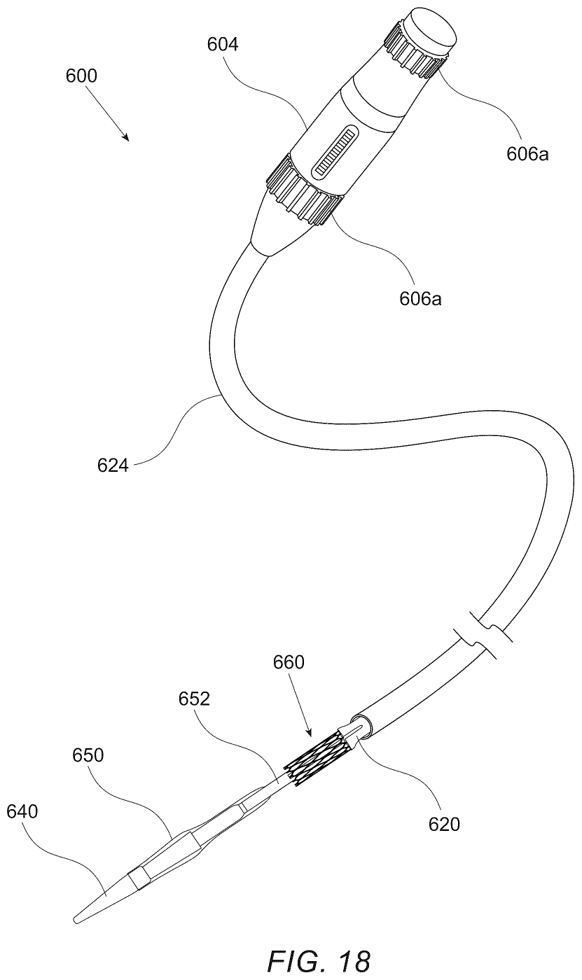

FIG. 1A illustrates a high-level perspective view of an exemplary prior art prosthetic valve 100. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by a delivery apparatus 600 (shown for example in FIG. 18) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prior art prosthetic valve 100 or any other prosthetic valve of the current disclosure (such as prosthetic valves 300, 400 or 1200 further disclosed herein) may include any prosthetic valve configured to be mounted within the native aortic valve, the native mitral valve, the native pulmonary valve, and the native tricuspid valve.

It is understood that the prosthetic valves disclosed herein may be used with a variety of delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus. Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165 and U.S. Provisional Application No. 63/085,947, filed Sep. 30, 2020, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

The prosthetic valve 100 has an inflow end 104 and an outflow end 106. In some instances, the inflow end 104 is the distal end of the prosthetic valve 100, and the outflow end 106 is the proximal end of the prosthetic valve 100. Alternatively, depending for example on the delivery approach of the valve, the inflow end can be the proximal end of the prosthetic valve, and the outflow end can be the distal end of the prosthetic valve.

The term “proximal”, as used herein, generally refers to a position, direction, or portion of a device or a component of a device, which is closer to the user (for example, during an implantation procedure) and further away from the implantation site.

The term “distal”, as used herein, generally refers to a position, direction, or portion of a device or a component of a device, which is further away from the user and closer to the implantation site.

The term “outflow”, as used herein, refers to a region of the prosthetic valve through which the blood flows through and out of the prosthetic valve 100, for example between the valve's central axis 30 and the outflow end 106.

The term “inflow”, as used herein, refers to a region of the prosthetic valve through which the blood flows into the prosthetic valve 100, for example between inflow end 104 and the valve's central axis 30.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

The prosthetic valve 100 comprises an annular frame 102 movable between a radially compressed state and a radially expanded state, and a leaflet assembly 130 mounted within the frame 102. The frame 102 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter (not shown), and then expanded inside a patient by an inflatable balloon or equivalent expansion mechanism. Alternatively or additionally, the frame 102 can be made of shape-memory materials such as, but not limited to, nickel titanium alloy (e.g., Nitinol). When constructed of a shape-memory material, the frame 102 can be crimped to a radially compressed state and restrained in the compressed state by insertion into an outer delivery shaft or equivalent mechanism of a delivery apparatus 600.

In the example illustrated in FIG. 1A, the frame 102 is an annular, stent-like structure comprising a plurality of intersecting struts 112, and defining a central axis 30. The frame 102 defines a frame inner surface 110 facing the central axis 30, and an opposite frame outer surface 108 facing away from the central axis 30, for example facing the surrounding anatomy, including annular and blood vessel walls, when implanted in a patient's body. In this application, the term “strut” 112 encompasses vertical struts, angled or curved struts, support posts, commissure windows, and any similar structures described by U.S. Pat. Nos. 7,993,394 and 9,393,110, which are incorporated herein by reference. A strut 112 may be any elongated member or portion of the frame 102. The frame 102 can have one or more multiple rows of cells defined by intersecting struts 112. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

Struts 112 comprise angled struts 114, and optionally vertical struts 116. The term “vertical strut” refers to a strut that generally extends in an axial direction, while the term “angled strut” generally refers to a strut that can extend at an angle relative to an axial line intersecting therewith along a plane defined by the frame 102. It is to be understood that the term “angled strut” encompasses both linear angled struts and curved struts. The exemplary frame 102 of the prosthetic valve 100 illustrated in FIG. 1A comprises a plurality of angled struts 114 and at least two types of vertical struts 116, namely outflow vertical struts 116a formed along the uppermost row of cells (i.e., adjacent outflow end 106), and inflow vertical struts 116b formed along the lowermost row of cells (i.e., adjacent inflow end 104).

Two or more struts 112 can intersect at junctions 120, which can be can be equally or unequally spaced apart from each other. The struts 112 may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

FIG. 18 illustrates a delivery apparatus 600, according to one configuration, adapted to deliver a balloon expandable prosthetic valve 660 described herein (e.g., prosthetic valve 100, 300, 400 and 1200). It should be understood that the delivery apparatus 600 can be used to implant prosthetic devices other than prosthetic valves, such as stents or grafts.

The delivery apparatus 600 includes a handle 604 and a balloon catheter 652 having an inflatable balloon 650 mounted on its distal end. The balloon expandable prosthetic valve 660 can be carried in a crimped state over the balloon catheter 652. Optionally, an outer delivery shaft 624 can concentrically extend over the balloon catheter 652, and a push shaft 620 disposed over the balloon catheter 652, optionally between the balloon catheter 652 and the outer delivery shaft 624.

The outer delivery shaft 624, the push shaft 620, and the balloon catheter 652, can be configured to be axially movable relative to each other. For example, a proximally oriented movement of the outer delivery shaft 624 relative to the balloon catheter 652, or a distally oriented movement of the balloon catheter 652 relative to the outer delivery shaft 624, can expose the prosthetic valve 660 from the outer delivery shaft 624. The delivery apparatus 600 can further include a nosecone 640 connected to the distal end of a nosecone shaft (hidden from view in FIG. 18) extending through a lumen of the balloon catheter 652.

Outer delivery shaft 624, push shaft 620, balloon 650, balloon catheter 652, and nosecone 640 along with the nosecone shaft can be formed from any of various suitable materials, such as nylon, braided stainless steel wires, or a polyether block amide (commercially available as Pebax®). In one example, outer delivery shaft 624 and push shaft 620 have longitudinal sections formed from different materials in order to vary the flexibility of the shafts along their lengths. In another example, the nosecone shaft has an inner liner or layer formed of Teflon® to minimize sliding friction with a guide wire (not shown).

The proximal ends of the balloon catheter 652, the outer delivery shaft 624, the push shaft 620, and optionally the nosecone shaft, can be coupled to the handle 604. During delivery of the prosthetic valve 660, the handle 604 can be maneuvered by an operator (e.g., a clinician or a surgeon) to axially advance or retract components of the delivery apparatus 600, such as the nosecone shaft, the balloon catheter 652, the outer delivery shaft 624, and/or the push shaft 620, through the patient's vasculature, as well as to inflate the balloon 650 mounted on the balloon catheter 652, so as to expand the prosthetic valve 660, and to deflate the balloon 650 and retract the delivery apparatus 600 once the prosthetic valve 660 is mounted in the implantation site.

The handle 604 can include a steering mechanism configured to adjust the curvature of the distal end portion of the delivery apparatus 600. In the illustrated example, the handle 604 includes an adjustment member, such as the illustrated rotatable knob 660a, which in turn is operatively coupled to the proximal end portion of a pull wire. The pull wire can extend distally from the handle 604 through the outer delivery shaft 624 and has a distal end portion affixed to the outer delivery shaft 624 at or near the distal end of the outer delivery shaft 624. Rotating the knob 660a can increase or decrease the tension in the pull wire, thereby adjusting the curvature of the distal end portion of the delivery apparatus 600. Further details on steering or flex mechanisms for the delivery apparatus can be found in U.S. Pat. No. 9,339,384, which is incorporated by reference herein. The handle 604 can further include an adjustment mechanism including an adjustment member, such as the illustrated rotatable knob 606b. The adjustment mechanism can be configured to adjust the axial position of the push shaft 620 relative to the balloon catheter.

The prosthetic valve 660 can be carried by the delivery apparatus 600 during delivery in a crimped state, and expanded by balloon inflation to secure it in a native heart valve annulus. In one exemplary implantation procedure, the prosthetic valve 660 is initially crimped over the balloon catheter 652, proximal to the inflatable balloon 650. Because prosthetic valve 660 is crimped at a location different from the location of balloon 650, prosthetic valve 660 can be crimped to a lower profile than would be possible if it was crimped on top of balloon 650. This lower profile permits the clinician to more easily navigate the delivery apparatus 600 (including crimped prosthetic valve 660) through a patient's vasculature to the treatment location. The lower profile of the crimped prosthetic valve is particularly helpful when navigating through portions of the patient's vasculature which are particularly narrow, such as the iliac artery.

The balloon 650 can be secured to balloon catheter 652 at its balloon proximal end, and to either the balloon catheter 652 or the nosecone 640 at its distal end. The distal end portion of the push shaft 620 is positioned proximal to the outflow end (e.g., outflow end 106, 306, 406 and 1206) of the prosthetic valve 660.

When reaching the site of implantation, and prior to balloon inflation, the push shaft 620 is advanced distally, allowing its distal end portion to contact and push against the outflow end of prosthetic valve 660, pushing the valve 660 distally therewith. The distal end of push shaft 620 is dimensioned to engage with the outflow end of the prosthetic valve 660 in a crimped configuration of the valve. In some implementations, the distal end portion of the push shaft 620 can be flared radially outward, to terminate at a wider-diameter that can contact the prosthetic valve 660 in its crimped state. Push shaft 620 can then be advanced distally, pushing the prosthetic valve 660 therewith, until the crimped prosthetic valve 660 is disposed around the balloon 650, at which point the balloon 650 can be inflated to radially expand the prosthetic valve 660. Once the prosthetic valve 660 is expanded to its functional diameter within a native annulus, the balloon 650 can be deflated, and the delivery apparatus 600 can be retrieved from the patient's body.

In particular implementations, the delivery apparatus 600 with the prosthetic valve 660 assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 660 and the delivery apparatus 600 can be free of any liquid. Methods for treating tissue leaflets for dry storage are disclosed in U.S. Pat. Nos. 8,007,992 and 8,357,387, both of which documents are incorporated herein by reference.

The leaflet assembly 130 comprises a plurality of leaflets 140 (e.g., three leaflets), positioned at least partially within the frame 102, and configured to regulate flow of blood through the prosthetic valve 100 from the inflow end 104 to the outflow end 106. While three leaflets 140 arranged to collapse in a tricuspid arrangement, are shown in the exemplary implementation illustrated in FIG. 1A, it will be clear that a prosthetic valve 100 can include any other number of leaflets 140. The leaflets 140 are made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

FIG. 1B shows a single representative leaflet 140, and FIG. 1C shows three separated leaflets 140 prior to attachment to the frame 102 and to each other, collectively defining the leaflet assembly 130, according to some examples. Each leaflet 140 has a rounded cusp edge 146 opposite a free edge 142, and a pair of generally oppositely-directed tabs 152 separating the cusp edge 146 and the free edge 142. The cusp edge 146 forms a single scallop and defines a cusp edge midpoint 148, opposite to a free edge midpoint 144 defined by the corresponding free edge 142.

An imaginary line shown as a continuation of the cusp edge toward the free edge along each tab 152, defines a tab hinge line 154, illustrated as a dashed line in FIG. 1B. It is to be understood that the term “free edge”, as used herein, refers to the upper edge of the leaflet extending between both tab hinge lines 154. That is to say, the free edge 142 has a total length Lp that does not include the tabs 152, as shown in FIG. 1B. The leaflet body 150 is defined as the portion of the leaflet 140 that extends between the cusp edge 146, tab hinge lines 154, and free edge 142. That is to say, leaflet body 150 does not include the tabs 152. Each leaflet body 150 defines two leaflet body sidelines 156, each extending from the cusp edge midpoint 148 to the free edge 142. For example, leaflet body sideline 156a shown in FIGS. 1B and 1C includes tab hinge line 154a and the portion of the cusp edge 146 extending therefrom up to the cusp edge midpoint 148, and leaflet body sideline 156b includes tab hinge line 154b and the portion of the cusp edge 146 extending therefrom up to the cusp edge midpoint 148.

The leaflet 140 defines a leaflet first face 158, that can be also referred to as a leaflet outer face, facing away from the central axis 30 when the leaflet assembly 130 is mounted in the frame 102, and a leaflet second face 160, that can be also referred to as a leaflet inner face, facing toward central axis 30 and toward the other leaflets 140 of the leaflet assembly 130 shown in FIG. 1C. Any of the leaflet first face 158 and/or second face 160 is a face defined over the leaflets body 150 and its tabs 152.

When the leaflets 140 are coupled to the frame and to each other, the lower edge of the resulting leaflet assembly 130 desirably has an undulating, curved scalloped shape. By forming the leaflets with this scalloped geometry, stresses on the leaflets 140 are reduced which, in turn, improves durability of the valve. The scalloped geometry also reduces the amount of tissue material used to form the leaflet structure, thereby allowing a smaller, more even crimped profile at the inflow end of the valve.

The leaflets 140 define a non-planar coaptation plane (not annotated) when their free edges 142 co-apt with each other to seal blood flow through the prosthetic valve 100. Leaflets 140 can be secured to one another at their tabs 152 to form commissure assemblies 132 of the leaflet assembly 130, which can be secured, directly or indirectly, to structural elements connected to the frame 102 or embedded therein, such as commissure posts or commissure windows 118. When secured to two other leaflets 140 to form leaflet assembly 130, the cusp edges 146 of the leaflets 140 collectively form the scalloped line of the leaflet assembly 130, shown as a dashed line in FIG. 1A.

In some examples, at least three some (e.g., three) of the outflow vertical struts 116a can be commissure support struts that can define axially extending window frame portions, also termed commissure windows 118. As shown in FIG. 1A, the commissure window 118 can be configured to receive a corresponding commissure assembly 132. For example, the tabs 152 of a commissure assembly 132 can be passed through the commissure window 118 outward along the radial direction of the frame 102 in order to secure the commissure assembly 132 to the frame 102.

Various configurations are known in the art by which commissure assemblies may be formed and coupled to a frame 102, some of which can include an outer wedge inserted into between the tabs once they are passed to the radially-outer side of the commissure window. The outer wedge can increase a width of the portion of the commissure assembly extending out of the commissure window (radially away from central axis 30) such that this outer portion of the commissure assembly cannot pass back through the commissure window.

FIG. 1A illustrates, in partially enlarged view, one exemplary prior art configuration of a commissure assembly 132 coupled to the commissure window 118 using an outer wedge 134. As shown, the commissure assembly 132 can be formed such that two tabs 152a and 152b of adjacent leaflets are wrapped over the outer wedge 134 at the portion extending radially away from the commissure window 118. The total width of both tabs 152 with the outer wedge 134 is greater than the width of the commissure window 118, thereby preventing, or at least restraining, passage of the outer portion of the commissure assembly 132 radially inward (i.e., toward central axis 30).

While the commissure window 118 is illustrated in FIG. 1A as an opening formed within an outflow vertical strut 116a of the frame 102, it is to be understood that such commissure windows can be similarly formed as part of other components attached to the frame 102, such as actuators of mechanically expandable prosthetic valves or other types of structural posts, and that other commissure assembly configuration can be formed without wedge members, for example by wrapping or otherwise suturing portions of the tabs to or around struts or other posts attached to the frame. Further details regarding prosthetic valves, including the manner in which commissures may be mounted to their frames, are described in U.S. Pat. Nos. 6,730,118, 7,393,360, 7,510,575, 7,993,394, 8,252,202, and 9,393,110; U.S. Publication Nos. 2018/0325665, 2019/0105153, U.S. Application Nos. 62/869,948 and 62/813,643; and PCT Application No. PCT/US2019/61392, all of which documents are incorporated herein by reference. Any of the techniques and mechanisms disclosed in the prior documents can be used to connect commissure assemblies 132, directly or indirectly, to the frame 102.

The prosthetic valve 100 can also include one or more skirts or sealing members. For example, the prosthetic valve 100 can include an inner skirt mounted on the frame inner surface 110 and/or an outer skirt mounted on the frame outer surface 108 (not shown in FIG. 1A). The inner skirt 122 can be a circumferential inner skirt that spans an entire circumference of the frame inner surface 110, having an inner having a skirt outer surface 124 facing the frame 102 and a skirt inner surface 126 facing central axis 30. The inner skirt 122 can function as a sealing member to prevent or decrease perivalvular leakage (e.g., when the prosthetic valve is placed at the implantation site) and as an attachment surface to anchor a portion of the leaflets 140 to the frame 102. In particular examples, the cusp edges 146 can be sutured to the inner skirt 122 along scallop line 128, which in turn can be sutured to selected struts of the frame. An outer skirt can function as a sealing member by sealing against the tissue of the native valve annulus and helping to reduce paravalvular leakage past the prosthetic valve 100. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame. Further details regarding the inner and outer skirts and techniques for assembling the leaflets to the inner skirt and assembling the skirts on the frame are disclosed in U.S. Provisional Application No. 62/854,702, U.S. Provisional Application No. 62/797,837 and U.S. Patent Application Publication No. 2019/0192296, each of which is incorporated herein by reference.

As mentioned above, a leaflet 140 is made of a flexible material that can be derived from a biological materials (e.g., pericardial tissue) or a bio-compatible synthetic material. For example, a portion of pericardium can be flattened over a flat surface and cut to form leaflet 140 shown in FIG. 1B. The cut portion of the pericardium is then bent such that the free edge 142 becomes curved inwards, approximating free edge midpoint 144 toward central axis 30, such that the distance between the tabs 152a, 152b, and in particular, the tab hinge lines 154a, 154b, is reduced. Although the region around free edge midpoint 144 becomes curved, the rest of free edge 142 can remain generally straight. Each leaflet 140 is flattenable, i.e. it can be unbent and flattened.

While FIG. 1C illustrates a high-level perspective view of the leaflet assembly 130 in an almost closed position, FIG. 1D illustrates a high-level perspective view of the leaflet assembly 130 in an open position and FIG. 1E illustrates a high-level cut-away perspective view of the leaflet assembly 130 in the open position. The leaflets 140 are illustrated in FIGS. 1D-1E without the tabs 152 for simplicity. Responsive to an increase in pressure at the inflow end 104 of frame 102, for example during the systole, leaflets 140 transition to the open position, as illustrated in FIGS. 1D-1E, thereby allowing blood to flow therethrough. As illustrated, in the open position, a lateral fold 162 is formed in each leaflet 140, extending thereacross, thereby creating a space 164 within lateral fold 162. The flow of blood over lateral fold 162 and space 164 can result in flow separation, due to excessive momentum loss immediately after flowing past lateral fold 162. This disrupts the flow with turbulence and flow eddies. The turbulence can also cause free edges 142 of leaflets 140 to flutter, and the stresses of this flutter can lead to fatigue failure over time.

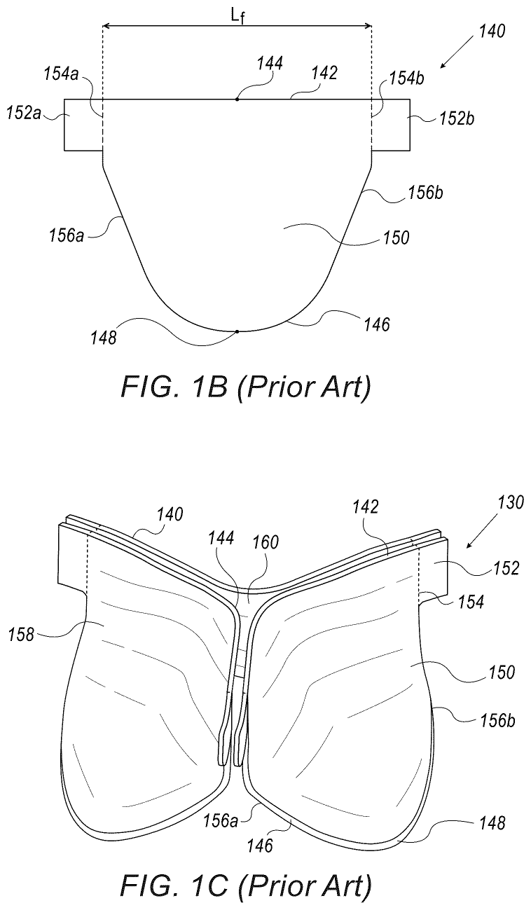

FIGS. 2A-2D illustrate various high-level perspective views of a leaflet 240 for a leaflet assembly 230 of a prosthetic valve, in accordance with some examples. In one example, leaflet 240 is made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, the entire contents of each of which are incorporated by reference herein.

Similar to leaflet 140 described above, with like reference numerals referring to like portions of the leaflet, leaflet 240 has: a leaflet first face 258; a leaflet second face 260 opposing leaflet first face 258; a free edge 242 extending between tab hinge lines 254 of tabs 252 and defining a free edge midpoint 244; a cusp edge 246 opposing free edge 242 and defining a cusp edge midpoint 248 opposing free edge midpoint 244; a leaflet body 250 defined between the cusp edge 246, tab hinge lines 254 and free edge 242; a first leaflet body sideline 256a extending from the cusp edge midpoint 248 to the free edge 242; and a second leaflet body sideline 256b opposing first leaflet body sideline 256a. The term “free edge”, as used herein, means an edge which is not connected to a frame, thereby allowing the free edge to move back and forth as blood flows through leaflet assembly 230 across leaflet 240.

In one example, leaflet 240 is not flattenable. The term “not flattenable”, as used herein, means that it cannot be flattened. Particularly, in one example, if an attempt is made to straighten out the curve of free edge 242 of leaflet 240, the curve will not be able to be completely straightened such that leaflet 240 becomes flat. In another example, if an attempt is made to straighten out the curves of first leaflet body sideline 256a and second leaflet body sideline 256b, the curves will not be able to be completely straightened such that leaflet 240 becomes flat. In one further example, each of first face 258 and second face 260 of leaflet 240 is a non-developable surface. Thus, first face 258 and second face 260 cannot be flattened. This is in contrast to leaflets 140, described above, that are flattenable.

In one example, leaflet 240 is curved from first leaflet body sideline 256a to second leaflet body sideline 256b. Particularly, in such an example, any cross-section of leaflet 240 extending from first leaflet body sideline 256a to second leaflet body sideline 256b defines a curve. In one further example, the curve defined by each cross-section of leaflet 240 extending from first leaflet body sideline 256a to second leaflet body sideline 256b exhibits a generally parabolic shape. In another further example, the curve of leaflet 240 from first leaflet body sideline 256a to second leaflet body sideline 256b forms a predetermined section of an ellipsoid.

In one example, leaflet first face 258 is a concave surface. In one further example, leaflet first face 258 is a concave curve whose first end is defined by first leaflet body sideline 256a and whose second end is defined by second leaflet body sideline 256b. The ends of a concave surface, as described herein, are defined such that the concavity of the surface extends from the first end to the second end thereof. In another further example, the concavity of leaflet first face 258 is three-dimensional, such that the concave surface is defined by a first concave cross-section extending from first leaflet body sideline 256a to second leaflet body sideline 256b and a second concave cross-section extending from free edge 242 to cusp edge midpoint 248.

In another example, leaflet second face 260 is a convex surface. In one further example, leaflet second face 260 is a convex surface whose first end is defined by first leaflet body sideline 256a and whose second end is defined by second leaflet body sideline 256b. The ends of a convex surface, as described herein, are defined such that the convexity of the surface extends form the first end to the second end thereof. In another further example, the convexity of leaflet second face 260 is three-dimensional, such that the concave surface defined by a first concave cross-section extending from first leaflet body sideline 256a to second leaflet body sideline 256b and a second concave cross-section extending from free edge 242 to cusp edge midpoint 248.

In one example, the distance between first leaflet body sideline 256a and second leaflet body sideline 256b decreases from free edge 242 to cusp edge midpoint 248, i.e. the distance is greater at free edge 242 than at cusp edge midpoint 248. In one further example, this distance is measured via the shortest path along leaflet first face 258 or leaflet second face 260. In another further example, this distance is measured via the shortest direct line from first leaflet body sideline 256a to second leaflet body sideline 256b.

In one example, each of first leaflet body sideline 256a and second leaflet body sideline 256b is curved. In one further example, each of first leaflet body sideline 256a and second leaflet body sideline 256b is curved from free edge 242 to cusp edge midpoint 248. In another example, first leaflet body sideline 256a and second leaflet body sideline 256b meet in cusp edge midpoint 248. In one further example, cusp edge midpoint 248 is defined by the meeting point of first leaflet body sideline 256a and second leaflet body sideline 256b. In another example, first leaflet body sideline 256a and second leaflet body sideline 256b form a continuous curve, with cusp edge midpoint 248 being a local extremum of this curve. In one further example, the ends of the continuous curve are defined by free edge 242.

In one example, as described above, leaflet 240 is curved from free edge 242 to the cusp edge midpoint 248. As illustrated in FIG. 2D, a curve 272 is defined between any point 241 on free edge 242 and the cusp edge midpoint 248. A curve 274 is further defined between the free edge midpoint 244 and the cusp edge midpoint 248. A curve 276 is further defined between free edge midpoint 244 and point 241. If curves 272, 274 and 276 are straightened out, they define a triangle 280, as illustrated in FIG. 2E. In other words, the sides of triangle 280 represent the arc lengths of curves 272, 274 and 276. In one example, when comparing the area of triangle 280 to the area of a triangle whose apexes are the equivalent points on flattenable leaflet 140, the area of triangle 280 is at least 1.5 times greater than the area of the equivalent triangle of leaflet 240. In one further example, the area of triangle 280 is at least 3 times greater than the area of the equivalent triangle of leaflet 240.

If curve 272 coincides with leaflet body sideline 256, the length of curve 276, extending along free edge 242 from free edge midpoint 244 to the leaflet body sideline 256, represent half of the arc length of the free edge 242. Thus, in such a case, the total arc length Lf of free edge 242 is equal to twice the length of curve 276. In one example, when comparing the arc length Lf of free edge 242 to the length of the free edge 142 of flattenable leaflet 140 (shown, for example, in FIG. 1B), the arc length Lf of free edge 242 is at least 1.2 times greater than the equivalent length of free edge 142 of flattenable leaflet 140. In one further example, the arc length Lf of free edge 242 is at least 1.5 times greater than the area of the equivalent length of free edge 142 of flattenable leaflet 140. In one further example, the arc length Lf of free edge 242 is at least 2 times greater than the area of the equivalent length of free edge 142 of flattenable leaflet 140.

In one example, the arc length of curve 272 decreases as the closer point 241 is to first leaflet body sideline 256a or second leaflet body sideline 256b, i.e. the arc length of curve 272 exhibits a positive correlation with the distance between point 241 and the closer one of first leaflet body sideline 256a and second leaflet body sideline 256b. Thus, in such an example, the arc length of curve 274 is greater than the arc length of any curve 272.

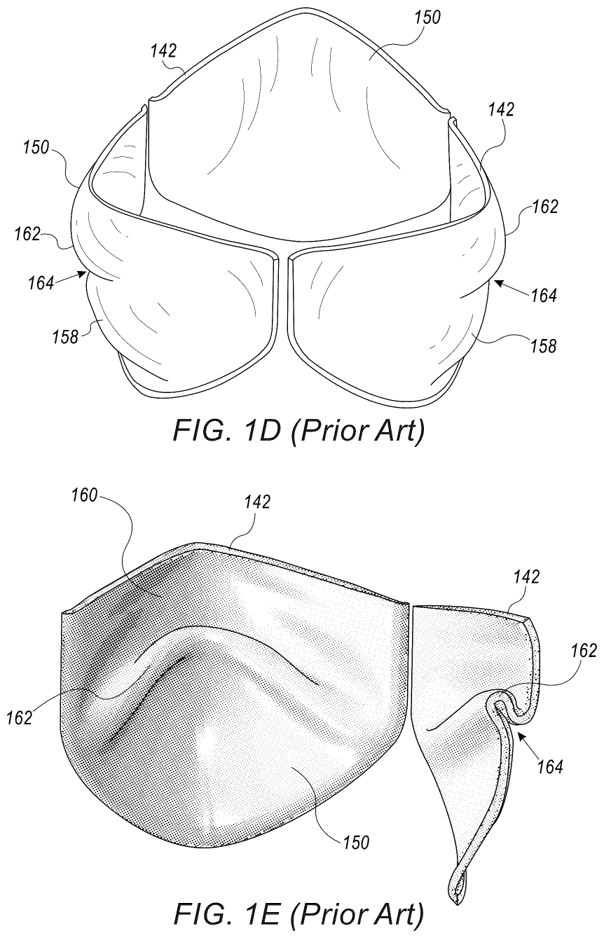

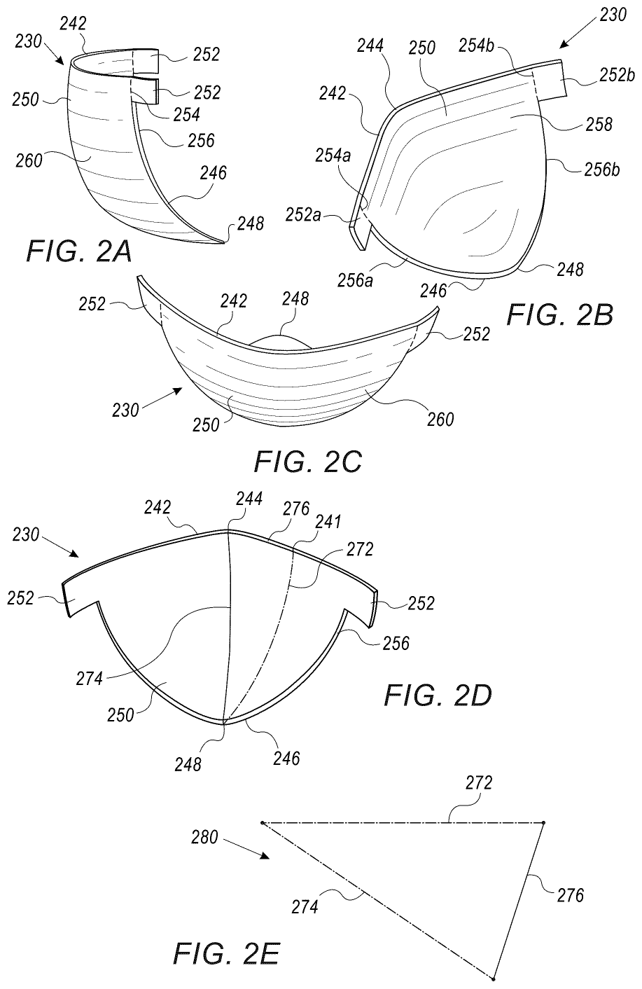

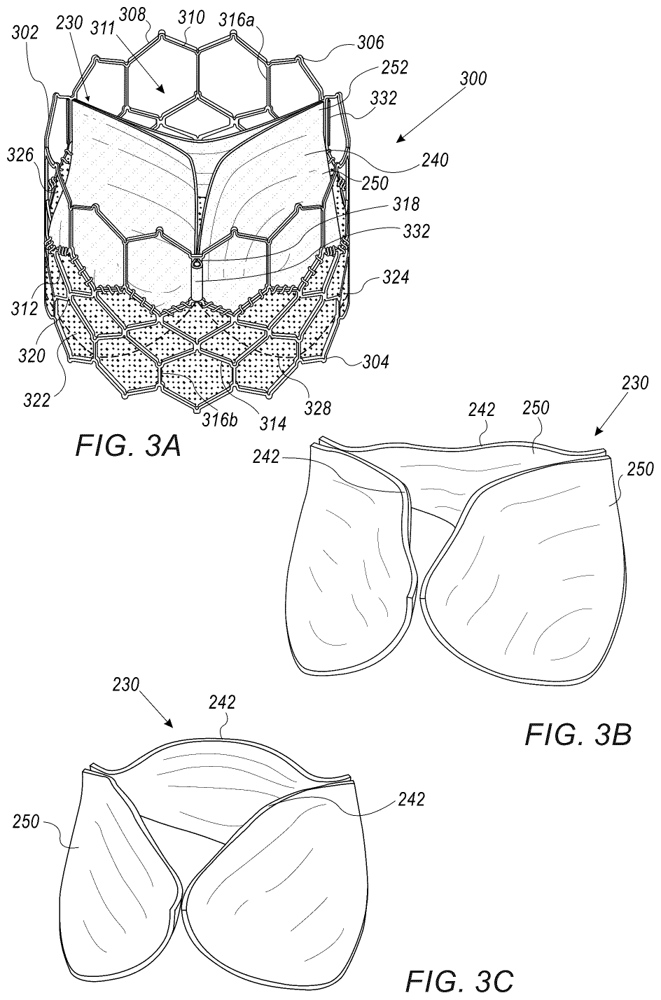

FIG. 3A illustrates a high-level perspective view of a prosthetic valve 300, in accordance with some examples. Prosthetic valve 300 is similar to prosthetic valve 100 with like reference numerals referring to like components thereof, and may be implemented according to any of the examples and optional configurations described above for prosthetic valve 100, except that it includes leaflet assembly 230 with non-flattenable leaflets 240 instead of leaflet assembly 130 with flattenable leaflets 140. In particular, prosthetic valve 300 comprises: a frame 302 having an inflow end 304 and an outflow end 306 and defining frame inner surface 310 and frame outer surface 308; with the leaflet assembly 230 mounted within frame 302. Three leaflets 240 are illustrated, however this is not meant to be limiting in any way. Particularly, while three leaflets 240 arranged to collapse in a tricuspid arrangement similar to the native aortic valve are illustrated, it will be clear that prosthetic valve 300 can include any other number of leaflets 240, such as two leaflets configured to collapse in a bicuspid arrangement similar to the native mitral valve, or more than three leaflets, depending upon the particular application.

FIGS. 3B-3D each illustrate a high-level perspective view of leaflet assembly 230 with leaflets 240, as they are positioned when mounted within frame 302 (removed from view in FIGS. 3B-3D for clarity). FIGS. 3B-3D show leaflets 240 gradually opening responsive to systolic blood flow through prosthetic valve 300, as will be described below. FIG. 3E illustrates a high-level perspective view of one and a half leaflets 240, in the open position shown in FIG. 3D. Leaflets 240 are shown without tabs 252 in FIGS. 3B-3E for simplicity. FIG. 3F illustrates a high-level cross-sectional view of a leaflet 240, in the open position shown in FIG. 3D. FIG. 3G illustrates a high-level perspective view of prosthetic valve 300, with leaflets 240 in the open position shown in FIG. 3D. FIG. 3H illustrates a high-level cross-sectional view of a portion of prosthetic valve 300, with leaflets 240 in the open position shown in FIG. 3D. FIGS. 3A-3H are described herein together.

As mentioned above, the term “prosthetic valve” refers to any type of a prosthetic valve deliverable to a patient's cardiac target site, which is radially expandable and compressible between a radially compressed, or crimped, state, and a radially expanded state. In one example, a prosthetic valve constructed differently than prosthetic valve 300 can be provided, without exceeding the scope of the disclosure. In one example, prosthetic valve 300 is constructed so as to be mounted within the native aortic valve, the native mitral valve, the native pulmonary valve and/or the native tricuspid.

In one example, as illustrated in FIGS. 3A and 3G, prosthetic valve 300 further comprises: an inner skirt 322 having a skirt outer surface 324 and a skirt inner surface 326; and a plurality of commissure assemblies 332. In another example (not shown), prosthetic valve 300 further comprises an outer skirt. In one example, frame 302 comprises a plurality of interconnected struts 312 that can be constructed according to any of the examples described hereinabove with respect to struts 112, and can include angled struts 314, vertical struts 316 that can comprise both outflow vertical struts 316a and inflow vertical struts 316, wherein at least some of the outflow vertical struts 316a can include commissure windows 318. With further similarity to any example described above for struts 112, struts 312 can intersect at junctions 120.

Frame 302, as well as inner skirt 322 and the outer skirt, can be similar in structure and function to any example described hereinabove with respect to frame 102, and in the interest of brevity will not be described further.

In one example, each adjacent pair of leaflets 240 are secured to each other at a respective commissure assembly 332. The commissure assemblies 332 can be similar in structure and function to any example described herein above with respect to commissure assembly 132 or any other configuration of commissure assemblies disclosed herein or known in the art, and may be similarly utilized to mount the leaflet assembly 230 to the frame 302 such that the leaflet assembly 230 is attached to the frame 302 at the outflow portions via commissure assemblies 332, and along the cusp edges 246 along scallop line 328 that can be optionally sutured to the inner skirt 322.

In one example, leaflets 240 and tabs 252 are formed of a continuous piece of material. In another example, separate leaflets 240 are provided, and are separately mounted within frame 302 and/or secured to each other.

Although prosthetic valve 300 is illustrated and described herein as comprising an inner skirt 322, this is not meant to be limiting in any way. In another example (not shown), inner skirt 322 is not provided. In another example, an outer skirt is not provided. In one example, inner skirt 322 is arranged to function as a sealing member to prevent or decrease perivalvular leakage.

In one example, leaflets 240 are secured to inner skirt 322 such that inner skirt 322 functions as an anchoring region for anchoring leaflets 240 to frame 302 and/or functions to protect leaflets 240 against damage which may be caused by contact with frame 302, for example during valve crimping or during working cycles of prosthetic valve 300. In one example, the cusp edge 246 of leaflets 240 secured to inner skirt 322 exhibits an undulating, curved and/or scalloped shape.

In one example, where an outer skirt is provided (not shown), the outer skirt is arranged to function as a sealing member retained between frame 302 and the surrounding tissue of the native annulus against which prosthetic valve 300 is mounted, thereby reducing risk of paravalvular leakage past prosthetic valve 300.

Frame 302 is constructed to form an enclosure 311 and leaflets 240 are positioned within enclosure 311. The term “enclosure”, as used herein, means an area that is surrounded by frame 302. In one example, frame 302 is generally cylindrical shaped. Inner skirt 322 is positioned within enclosure 311 and skirt outer surface 324 is juxtaposed with frame 302 such that skirt inner surface 326 faces enclosure 311.

Responsive to flow (such as systolic blood flow, or alternatively, any other fluid flow in an in-vitro setup) through enclosure 311, leaflets 240 begin to open, away from each other and towards frame 302. The gradual opening of leaflets 240 is illustrated by FIGS. 3B-3D. As leaflets 240 open, a bulge 262 is formed in leaflet second face 260. Bulge 262 extends between a bulge outflow end 264a, a bulge inflow end 264b, a bulge first side 266a and a bulge second side 266b. In one example, bulge first side 266a and bulge second side 266b each define a concave curve in a respective section 268 of leaflet second face 260. Thus, concave curved sections 268 are between bulge 262 and the respective one of first leaflet body sideline 256a and the second leaflet body sideline 256b.

In one example, the distance between bulge outflow end 264a and free edge midpoint 244 is greater than the distance between bulge inflow end 264b and the cusp edge midpoint 248. In another example, bulge inflow end 264b reaches the cusp edge midpoint 248.

In one example, the width of bulge 262 decreases from bulge outflow end 264a to bulge inflow end 264b, i.e. the width at bulge outflow end 264a is greater than the width at bulge inflow end 264b. The width of bulge 262 is defined as the distance between bulge first side 266a and bulge second side 266b. In another example, the maximum width of bulge 262 is 1/9 to ⅓ of the maximal distance between first leaflet body sideline 256a and second leaflet body sideline 256b. In one further example, the maximal distance between first leaflet body sideline 256a and second leaflet body sideline 256b is defined at free edge 242. In one example, the height to width ratio of bulge 262 is between 1:1 to 4:1. In one further example, the height of bulge 262 is defined as the distance between an apex of bulge 262 and the bases of curved sections 268. In one example, the surface of bulge 262 is essentially smooth. The term “smooth”, as used herein, means lacking folds, protrusions or indentations.

In one example, bulge 262 is formed in a central portion of leaflet 240, i.e. the distance between bulge first side 266a and first leaflet body sideline 256a is essentially equal to the distance between bulge second side 266b and second leaflet body sideline 256b. In another example, bulge 262 forms a corresponding indent 270 in leaflet first face 258, the shape of indent 270 being an inverse shape of bulge 262.

Due to the formation of bulge 262, the blood flow is not hindered by flow separation zones and eddy formations as described above in relation to flattenable leaflets 140. In turn, this prevents fluttering of free edges 242 of non-flattenable leaflets 240. Additionally, it is contemplated that the protrusion of bulge 262 into enclosure 311 provides improved stabilization of leaflet 240 and the blood flow pattern. This could result in an effective opening area (EOA) that is greater than the EOA provided by flattenable leaflets 140. Furthermore, indent 270 distances the central region of leaflet 240 away from frame 302, adding further stability to leaflet 240.

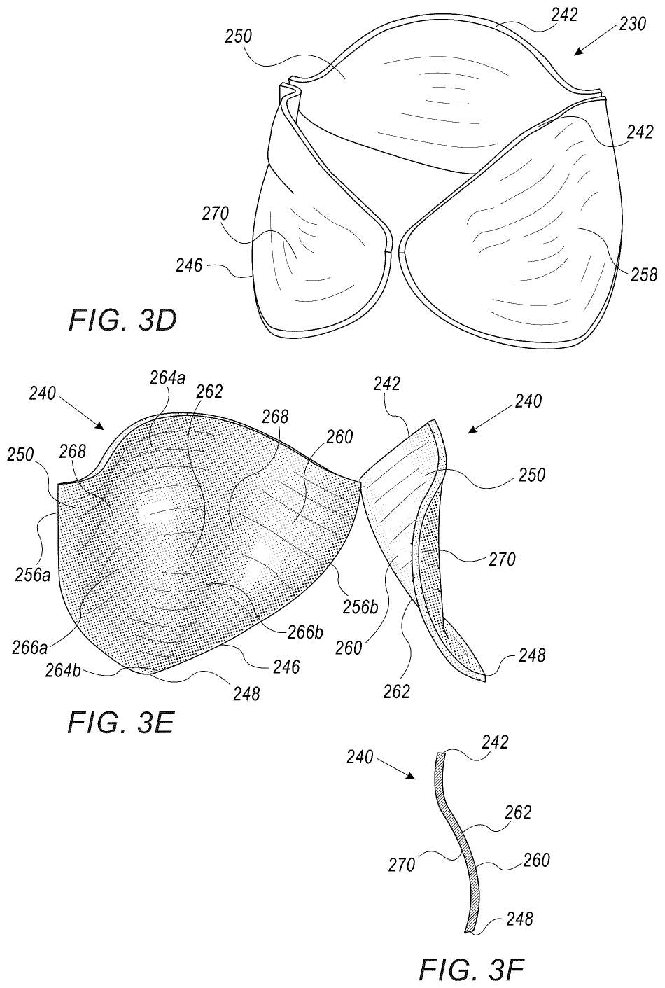

FIG. 4A illustrates a high-level perspective view of leaflet 240 and FIG. 4B illustrates a high-level perspective view of leaflet 240, showing the contour of leaflet 240 in a completely open position of the leaflet assembly 230, as described above. Particularly, FIGS. 4A-4B further illustrate: a curve 274, as described above; a plurality of curves 278 extending from curve 274 to second leaflet body sideline 256b; a plurality of lines 282 extending from the intersections of curves 278 and curve 274; a plurality of curves 284 extending from curves 278, at second leaflet body sideline 256b, to respective lines 284, away from leaflet first face 258; and a line 286 representing where frame 302 is in relation to leaflet 240, as described above in relation to FIGS. 3A-3F. Particularly, line 286 meets cusp edge midpoint 248.

Leaflet 240 is illustrated in FIGS. 4A and 4B in the almost closed position, as illustrated and described above in FIG. 3A. The points where lines 282 and curves 284 meet are the points where the contour of leaflet 240 reaches in a fully open position of the leaflet assembly 230, as illustrated and described above in FIG. 3G. As illustrated, very little space is left between frame 302 (represented by line 286) and leaflet 240 when open, thus maximizing the space between leaflets 240 and increasing the EOA. Additionally, as illustrated, the lower half of leaflet 240 reaches frame 302. It is contemplated that this aids in preventing lateral folds (162) from forming in leaflet 240.

The terms “open position of the leaflets” and “open position of the leaflet assembly”, as used throughout this specification, are interchangeable Similarly, the terms “closed position of the leaflets” and “closed position of the leaflet assembly”, as used throughout this specification, are interchangeable.

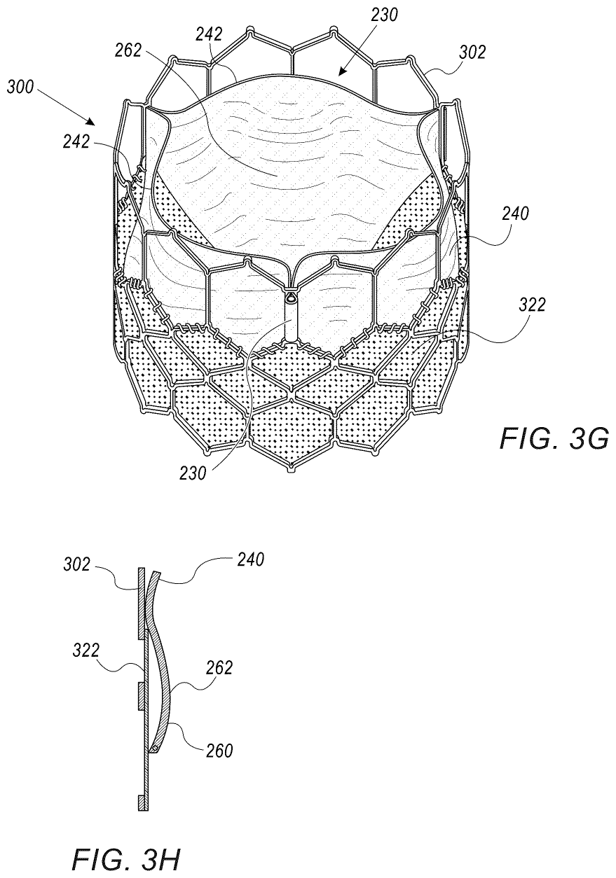

FIG. 5A illustrates a high-level perspective view of a prosthetic valve 400, in accordance with some examples. FIG. 5B illustrates a high-level side view of prosthetic valve 400, in accordance with some examples. FIG. 5C illustrates a high-level cross-sectional view of a portion of prosthetic valve 400, in accordance with some examples. FIGS. 5A-5C are described herein together.

Prosthetic valve 400 is in all respects similar to prosthetic valve 300, with the exception that leaflets 240 exhibit a lower center of coaptation 490. The term “center of coaptation”, as described herein, means a single point which is closest to free edge midpoint 244 of each leaflet 240 when leaflet assembly 230 is in the closed position. The suturing of leaflets 240 to inner skirt 422 along scallop line 428 is shown by dotted lines in FIG. 5B for clarity. The frame 402, as well as inner skirt 322 and the optional outer skirt, can be similar in structure and function to any example described hereinabove with respect to frames 102 and 302, and in the interest of brevity will not be described further. A commissure assembly 432 extends between a commissure assembly outflow end 431, which is closer to the outflow end 406 of the frame 402, and a commissure assembly inflow end 433, which is closer to the inflow end 404 of the frame 402, and can be similar in structure and function to any example described herein above with respect to commissure assembly 132 or any other configuration of commissure assemblies disclosed herein or known in the art. In one example, a commissure outflow plane 492 is defined by commissure assembly outflow ends 431, a commissure inflow plane 493 is defined by commissure assembly inflow ends 433, and a cusp midpoint plane 494 is defined by cusp edge midpoints 248 of leaflets 240.

In one example, center of coaptation 490 is located between commissure assembly inflow ends 433 and inflow end 404 of frame 402. In one further example, center of coaptation 490 is located between plane 493 and inflow end 404 of frame 402. In another further example, the distance of center of coaptation 490 to plane 492 is greater than a third of the distance between plane 492 and plane 494. In other words, center of coaptation 490 is less than ⅔ of the height of leaflets 240. In one example, such a position of center of coaptation 490 is achieved by increasing the curvature of leaflet 240 in the direction of inflow end 404. In one further example, for each leaflet 240, free edge midpoint 244 is closer to the height of cusp edge 246 due to the concavity of free edge 242.

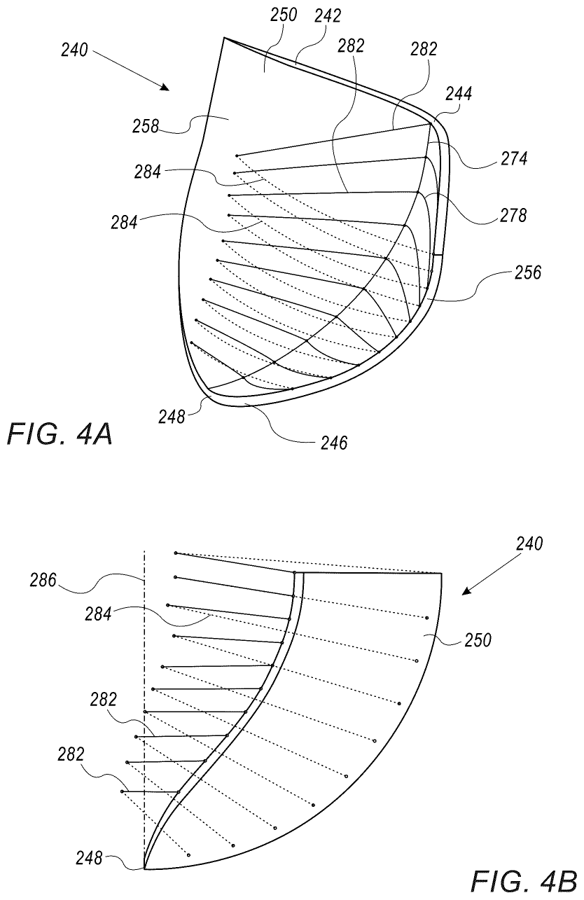

In another example, as illustrated in FIGS. 6A-6B, the curvature of leaflet 240 is deepened such that a portion of leaflet 240 extends past plane 494 towards inflow end 404 when leaflet assembly 230 is in the closed position. FIG. 6A illustrates a high-level perspective view of leaflet 240 and FIG. 6B illustrates a high-level cross-sectional view of leaflet 240.

Advantageously, due to the position of center of coaptation 490, the main recirculation area of the blood covers a significantly increased area between leaflets 240 and frame 402. As a result, adequate washout of blood is achieved, thereby reducing the risk of thrombus formation.

In some cases, for example when the prosthetic valve (400) is serves to replace the native aortic valve, upper or outflow cells of the frame may be facing the coronary ostia. In some instances, a patient may require implantation of a coronary stent of other procedure that requires access to a coronary artery after prosthetic valve implantation. For such instances, a physician may need to access the coronary artery through an opening defined by a cell facing the coronary ostium. Advantageously, lowering the center of coaptation 490 further serves to distance the upper portion of the leaflets away from the upper row of cells of the frame, so as to leave a larger portion of the upper cells exposed at all times in a manner that can improve perfusion to the coronary arteries through such cells, for example during diastole, and provide adequate access to the coronary arteries therethrough when required.

This advantage is not limited only to aortic replacement valves, but for prosthetic valves that can be implanted in other regions. For example, when a prosthetic valve 400 is implanted within a native mitral valve, some of the cells near the outflow end are facing the left ventricular outflow tract (LVOT). In such cases, lowering the center of coaptation 490 similarly serves to distance the outflow portion of the leaflets away from the outflow cells, so as to leave a larger portion of such cells exposed at all times in a manner that can retain adequate blood flow therethrough toward the LVOT.

A prosthetic valve can be manufactured in various sizes to accommodate variation in patients' native anatomy. The “size” of a prosthetic valve can correspond to its nominal expanded diameter. For example, a prosthetic valve configured for native aortic valve implantation can be manufactured in 20 mm, 23 mm, 26 mm, and 29 mm sizes. In other examples, a prosthetic valve can be manufactured in various other sizes.

Some prosthetic valves are configured such that each valve size can be expanded to a range of diameters (e.g., using a balloon or mechanically-assisted expansion mechanism). For example, a prosthetic valve with a nominal diameter of 23 mm (also referred to as a “23 mm valve”) can be expanded to diameters within the range of 20-26 mm in some cases or to diameters within a range of 22-24 mm in other cases. A prosthetic valve having an expanded diameter less than its nominal diameter is referred to herein as “under expanded”. For example, a 23 mm valve expanded to a 22 mm diameter is under expanded. A prosthetic valve expanded to its nominal diameter is referred to herein as “nominally expanded”. For example, a 23 mm valve expanded to a 23 mm diameter is nominally expanded. A prosthetic valve having an expanded diameter greater than its nominal diameter is referred to herein as “over expanded”. For example, a 23 mm valve expanded to a 24 mm diameter is over expanded.

Prior to or as a part of the delivery procedure, a prosthetic valve size must be selected. Prosthetic heart valve size can impact several factors, including the prosthetic valve's ability to resist migration relative to the native annulus and/or the impact the prosthetic valve has on the native tissue. For example, a prosthetic valve that is too small may migrate from the annulus and embolize. A prosthetic valve that is too large may damage native tissue if it is fully expanded or may not properly function if it is grossly under expanded (e.g., the leaflets may not fully open and/or will contact the frame when the leaflets open).

The size of the prosthetic valve can also impact blood flow characteristics, such as the pressure gradient across the prosthetic valve. Pressure gradient across the prosthetic valve is directly proportionate to the amount of force necessary to move blood from one side of the valve (e.g., the left ventricle) to the other (e.g., into the aorta). As such, a lower pressure gradient across the prosthetic valve is desirable because it can reduce the amount of work the heart must perform for adequate circulation.

A prosthetic valve according to any example herein, such as prosthetic valve 100 shown in FIG. 1A, as well as any of the prosthetic valve 300 or 400, has a nominal height H and a nominal radius R (see FIG. 1A), which are the height and radius of the prosthetic valve when nominally expanded. A conventional flattenable leaflet 140 will usually have a length Lf of its free edge 142 which is about 2R, which is a length selected to ensure adequate coaptation during the closed position of the leaflet assembly 130, especially for prosthetic valves having a nominal height H which is less than 2R. For example, a 26 mm valve will have a nominal radius R of about 13 mm, with leaflets 140 having length Lf of about 26 mm Since the perimeter of the frame 102 is alt, the perimeter of the leaflets 140 in the open position of the leaflet assembly 130, which is about 6R, results in the free edges 142 of the leaflets 140 being in very close proximity to the frame inner surface 110 in their open position when the prosthetic valve 100 is nominally expanded, and to contact the frame inner surface 110 if the prosthetic valve is under expanded.

An important design criterion of a prosthetic valve is to prevent or minimize contact between the movable portions of the leaflets (140) and the frame inner surface (110). Repeated contact between the movable portions of the leaflets and the metal frame during operation of the prosthetic valve can cause premature wear and eventual failure of the leaflets. Typically, the leaflets (140) are attached to the frame (102) in such a manner that the articulating or coaptation edges of the leaflets are spaced radially inward of the frame to prevent leaflet abrasion when the leaflets open under the flow of blood. FIG. 1A shows, in a zoomed in view, one exemplary commissure assembly 132 in which a portion of the leaflets 140 are coupled to each other along a distance Ma from the frame inner surface 110, such that the articulation line 135 of the free edges 142 is distanced radially inward away from the frame 102. While such configurations may allow the prosthetic valve 100 to be implanted in a wider range of patient annulus sizes, with reduced risk of the leaflets 140 contacting the frame 102 when the prosthetic valve 100 is under expanded, this also reduced the EOA and increases pressure gradient across the valve.

A significant advantage of utilization of non-flattenable leaflets 240 is the ability to provide such leaflets 240 with a longer arc length Lf of their free edges 242, resulting in a lower center of coaptation 490 as described above, while preserving adequate coaptation in a closed position of the leaflet assembly 230. Providing leaflets 240 with an arc length Lf which is significantly greater than 2R, particularly for prosthetic valves having a nominal height H which is less than 2R, enables the center of coaptation 490 to be lowered such that the leaflets 240 properly coapt in the closed position, without the risk of contacting the frame 402 in the open position, even when the prosthetic valve 400 is significantly under expanded. Thus, a greater arc length Lf of the free edges 242, resulting in a lower center of coaptation 490, enables the prosthetic valve 400 to be utilized in a much wider range of patient annulus sizes, without the need to offset the articulation line of the leaflets inward, thus preserving EOA and improving pressure gradient across the prosthetic valve. In some examples, the arc length Lf of each free edge 242 is greater than 2.2R. In some examples, the arc length Lf of each free edge 242 is greater than 2.5R. In some examples, the arc length Lf of each free edge 242 is greater than 3R.

Conventional commissure assemblies, such as, but not limited to, commissure assembly 132 shown in FIG. 1A, are assembled such that both adjacent leaflets 140 are in contact with each other at any time at the level of the frame inner surface 110 (or the inner surface of any other structure to which the commissure assembly is mounted), regardless of whether the leaflet assembly (130) in the closed or open positions. While FIG. 1A shows an exemplary commissure assembly 132 having both leaflets 140a, 140b coupled to each other along a distance Ma extending radially inward from the frame inner surface 110, in other configurations, the leaflets (140a, 140b) will not necessarily be coupled to each other along a distance Ma, yet the portion of the leaflets which extend through the commissure window (118), for example, will still be pressed against each other such that the leaflets are still in direct contact with one another at the portion immediately extending from the commissure window inward.

The close proximity between these portions of the leaflets 140 can cause tissue ingrowth and pannus formation over time, post implantation, which will result in a stiffened portion of the leaflets extending further radially inward, thereby reducing EOA and increasing pressure gradient across the prosthetic valve. For example, while a portion of the leaflets 140a, 140b are already coupled to each other along a distance Ma in the example illustrated in FIG. 1A, pannus formation overtime may cause this portion to extend further radially inward, distancing the articulation line 135 closer toward central axis 30, thereby reducing EOA beyond the originally intended value. Similarly, for commissure assemblies in which the articulation line 135 is closer to the frame inner surface 110 (that is to say, commissure assemblies that do not include a length Ma along which both leaflets are coupled to each other), pannus formation over time will result in creation of a stiffened region that, in effect, will be similar to the coupling along distance Ma shown in FIG. 1A, effectively distancing the articulation line 135 radially inward in a similar manner.

It may be desirable, thus, to provide commissure assemblies that do not encourage pannus formation (or other type of tissue overgrowth) at the region of the leaflets extending radially inward from the commissure support member (such as commissure window 118). In some examples, there is provided a commissure assembly comprising a separator positioned between portions of the leaflets extending through a commissure window, configured to separate between both leaflets at least at the level of the inner surface of the commissure window.

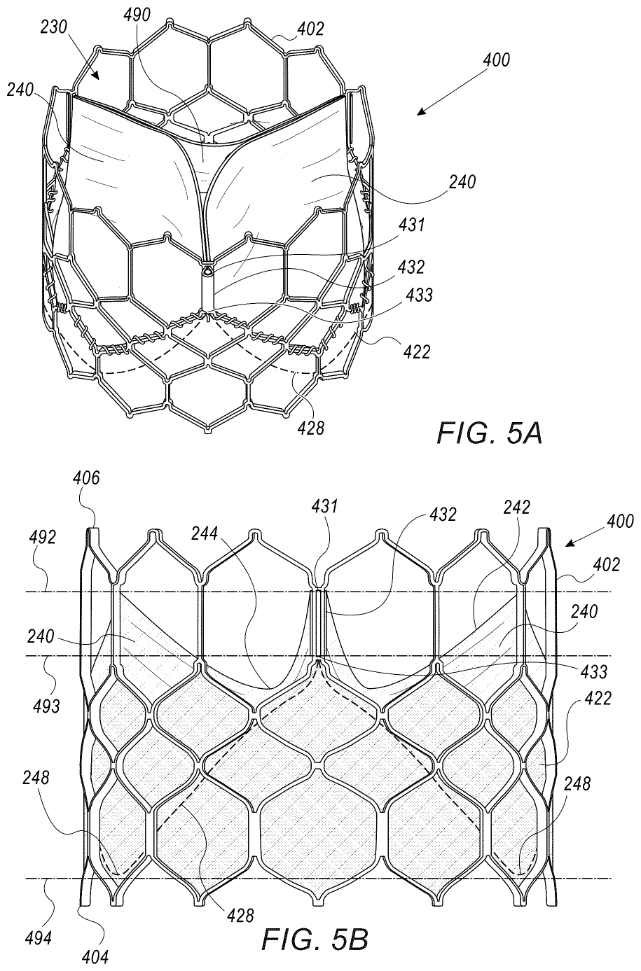

FIGS. 7A-7B show a partial top view and a partial perspective view of an exemplary commissure assembly 730 mounted in a commissure window 510. Commissure window 510 can be any representative commissure window, including commissure window formed as part of the frame, such as any of the frames 102, 302, 402 or any other frame known in the art, structured in a similar manner to that shown for commissure window 118 shown in FIG. 1A for example. Alternatively, commissure window 510 can be formed in a commissure support member which is not formed as an integral portion of the frame, but may be rather attached to the frame, such as commissure windows that may be formed in separate post member or actuators of mechanically expandable valves. Moreover, a commissure window 510 may be provided as a closed window, such as the commissure window 118 illustrated in FIG. 1A, or an open window, having its upper end exposed in a manner that allows leaflets to be slidably inserted thereinto from above.

Commissure window 510 comprises an opening defined between two sidewalls 512, and defines a commissure window inner surface 514 facing central axis 30, and commissure window outer surface 516 facing away from central axis 30. When the commissure window 510 is implemented as a part of the frame, such as commissure window 118 illustrated in FIG. 1A to be formed a type of an outflow vertical strut 116a of frame 102, the commissure window inner surface 514 can be at the level of the frame inner surface 110, and the commissure window outer surface 516 can be at the level of the frame outer surface 108.

A commissure assembly with a separator according to any of the examples disclosed herein, comprises portion of two adjacent leaflets 520a, 520b, including tabs 522a, 522b thereof, wherein each leaflet 520 has a leaflet and tab first face 524, which is the surface facing away from central axis 30 at the portions of the leaflets 520 positioned inside the frame (i.e., between the frame inner surface and the central axis), and an opposite leaflet and tab second face 526, which is the surface facing central axis 30 at the portions of the leaflets 520 positioned inside the frame. Leaflets 520 can be representative of any type of leaflets utilized in any type of a prosthetic valve, include flattenable leaflets 140 or non-flattenable leaflets 240 according to any of the examples disclosed herein.

A commissure assembly mounted in a commissure window 510 will include a portion of the leaflets 520a, 520b, and more specifically—portion of their tabs 522a, 522b, extending through the commissure window 510, such that the leaflet and tab first face 524 of both tabs 522 contact the sidewalls 512 of the commissure window 510. The commissure window 510 has an opening width Wc defined between both sidewalls 512, dimensioned to accommodate both leaflets 520, each leaflet 520 having a leaflet thickness Tl (which is the same thickness for both the leaflet body and the tab 522).

FIGS. 7A-7B show one example of a leaflet separator 740 disposed between a portion of the leaflets 520 that extends through the commissure window 510, such that the leaflet and tab second face 526 of each of the leaflet 520 directly contacts the separator 740. Separator 740 has a separator inner end 742 facing central axis 30, an opposite separator outer end 743 facing away from central axis 30, and a separator thickness Ts defined in the circumferential direction (i.e., perpendicular to the radial direction). In some cases, leaflet 520 can be made of a compressible material (e.g., pericardial tissue) that can be squeezed if needed to a width which is somewhat thinner than its un-squeezed or free-state with. For example, if the opening width We of the commissure window 510 is equal to about twice the leaflet thickness Tl (i.e., We=2T1), a separator (740) having a separator thickness Ts can be inserted between both leaflets 520 in a manner that somewhat squeezes them between the separator and sidewalls 512. The terms “leaflet separator” and “separator”, as used herein, are interchangeable.

FIGS. 7A-7B illustrate a specific exemplary configuration of a commissure assembly 730 which is somewhat similar to that of the commissure assembly 132 described above with reference to FIG. 1A, comprising an outer wedge 734, which can be similar to outer wedge 134, formed such that the portions of tabs 522a and 522b are wrapped over the outer wedge 734 at the portions extending radially out of commissure window outer surface 516. The total width of both tabs 522a, 522b (i.e., 2T1) with the outer wedge 134 is greater than the opening width Wc of the commissure window 510, thereby preventing, or at least restraining, passage of the outer portion of the commissure assembly 730 radially inward (i.e., toward central axis 30).

One or more sutures 736 can be employed to attached different portion of the commissure assembly 730 to each other. For example, a suture 736a can be stitched through both tabs 522a, 522b and the outer wedge 734 to couple them to each other at the portion of the commissure assembly 730 extending our of the commissure window 510. Similarly, sutured 736b and 736c can be stitched through tabs 522a, 522b and the separator 740, for example in the vicinity of the separator outer end 743 and inner end 742. It is to be understood that any other number and position of sutures or other coupling members can be employed, and that the configuration of two tabs wrapped over an outer wedge is shown merely for illustrative purposes, while commissure assemblies can be formed in any other manner known in the art, modified to further include a separator (740) as disclosed herein.

FIGS. 7A-7B show the leaflets 540 in their open position, having their free edges extending away from each other, wherein the separator 740 ensures that both leaflets 520a, 520b are distanced away from each other by a distance equal to its thickness Ts in this position, at least at the level of the separator inner end 742 or the commissure window inner surface 514, allowing blood flow between the leaflets 520 at this region in the open position. This separation during each phase of the open position (e.g., during systole in the case of implantation at the native aortic valve) allows the blood flow to wash against the leaflet second face 526 in this region, advantageously preventing stasis and reducing likelihood of tissue ingrowth and pannus formation at this region over time. The height of the separator 740 can be generally similar to the height of the tabs 522 at the level of the commissure window inner surface 514.

The separator (740) can be provided either as a rigid wedge member, for example plate-like member, or a flexible member, for example comprising fabric of polymeric flexible material. In some examples, the separator (740) is made of a thromboresistant material, or is coated with a thromboresistant materials, adapted to prevent tissue ingrowth and formation of pannus therearound. The term “thromboresistant”, as used herein, refers to a material's resistance to platelet adhesion and subsequent thrombus formation and/or tissue ingrowth in vitro and/or in vivo. In some examples, the separator (740) comprises thermoplastic polyurethane (TPU), including examples in which the separator (740) is made of TPU or examples in which the separator (740) is made of another soft or rigid material, coated by TPU.

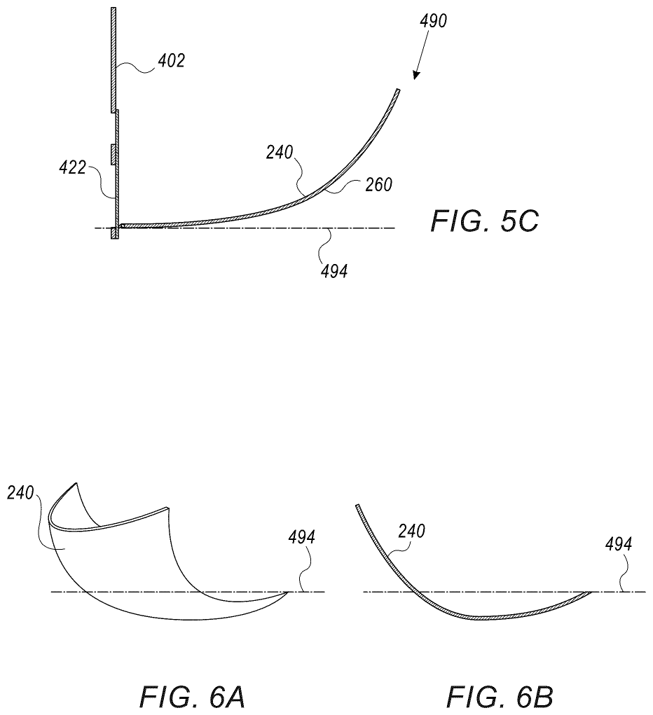

FIGS. 8A-8B illustrate various examples of the separator 740, shown in conjunction with commissure assembly 730 in a closed position of the leaflets 520. In the closed position, as shown, the leaflets 520a, 520b coapt such that they are pressed against each other radially inward to the separator inner end 742. Due to the separator thickness Ts, both leaflets 520a, 520b do not contact each other immediately after the separator inner end 742, but rather form am enclosed space 790, which can take the form of a triangularly-shaped space 790, extending from the separator inner end 742 up to the point of contact between the leaflets 520 in the closed position. Blood backflow may occur through these spaces 790 in the closed position to some extent. While a certain amount of backflow may be permissible without significantly affecting hemodynamic performance of the prosthetic valve, large spaces are to be avoided to prevent significant regurgitation.

The separator thickness Ts can be selected so as to provide sufficient distancing between adjacent leaflets 520a, 520b to allow blood flow therebetween at the level of the separator inner end 742 or the commissure window inner surface 514 in the open position of the leaflets 520, so as to wash over their second face 526 at this region in a manner that can reduce the likelihood of tissue ingrowth and pannus formation. On the other hand, separator thickness Ts directly influences the size of spaces 790 formed in the closed position, and should not exceed an upper threshold to avoid significant regurgitation. In some examples, the separator thickness is in the range of 100μ and 1 mm A minimal threshold of a 100μ may be sufficient to prevent pannus formation, while a maximal threshold of 1 mm may serve as an upper threshold beyond which the space 790 may result in significant regurgitation. It has been observed by the inventors that in certain implementations, a minimal distance of 300μ results in desired prevention of tissue ingrowth. In some examples, the separator thickness is in the range of 200μ and 500μ. In some examples, the separator thickness is in the range of 200μ and 400μ. In some examples, the separator thickness is in the range of 250μ and 350μ. In some examples, the separator thickness is at least 300 μ.

The length of the separator 740 is defined between its inner end 742 and out end 744, and may be similar or different than the radial length of the commissure window 510 (which can be, in some implementation, the thickness of the frame) defines between its inner 514 and outer 516 surfaces. FIG. 8A shows one exemplary implementation of a separator 740 having a radial length which is substantially similar to the distance between the inner 514 and outer 516 surfaces of the commissure window 510, wherein the separator outer end 743 is shown to be substantially at the level of the commissure window outer surface 516. FIG. 8B shows another exemplary implementation of a separator 740 having a shorter radial length (compared to the implementation of FIG. 8A, for example), wherein the separator outer end 743 is shown to be offset radially inward relative to the commissure window outer surface 516.

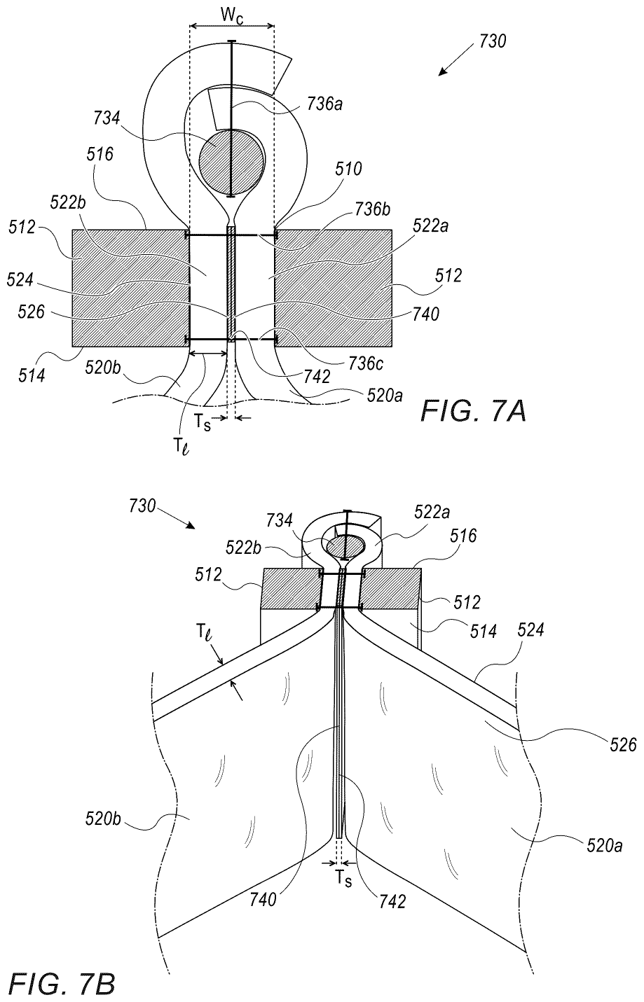

FIGS. 9A-9C illustrate various examples of the position of separator inner end 742 with respect to the commissure window inner surface 514. In some examples, the separator inner end 742 can be at the level of the commissure window inner surface 514, as shown in FIG. 9A. In other examples, a radial distance Ms can be defined between the separator inner end 742 and the level of the commissure window inner surface 514. FIG. 9B shows an example in which the separator 740 protrudes radially inward, having its inner end 742 positioned at a distance Ms radially inward to the level of the commissure window inner surface 514. FIG. 9C shows an example in which the separator 740 terminated within the commissure window 510 before reaching the inner surface 514, such that its inner end 742 is positioned at a distance Ms radially outward to the level of the commissure window inner surface 514. The distance Ms is preferably not greater than the separator thickness Ts, regardless of whether oriented radially inward or outward relative to inner surface 514. In some examples, the radial distance between the separator inner end 742 and the commissure window inner surface 514 is not greater than the separator thickness Ts, in either direction.

Some types of commissure assemblies can also include a coupling member, such as a flexible cloth or fabric, disposed around external surfaces of the leaflets, and in particular, of their tabs. Such coupling members can serve to protect portions of the leaflets from abrasion by interaction with the commissure window, and may assist in providing reliable attachment of the tabs to each other and/or to additional components of the commissure assembly, such as the outer wedge.

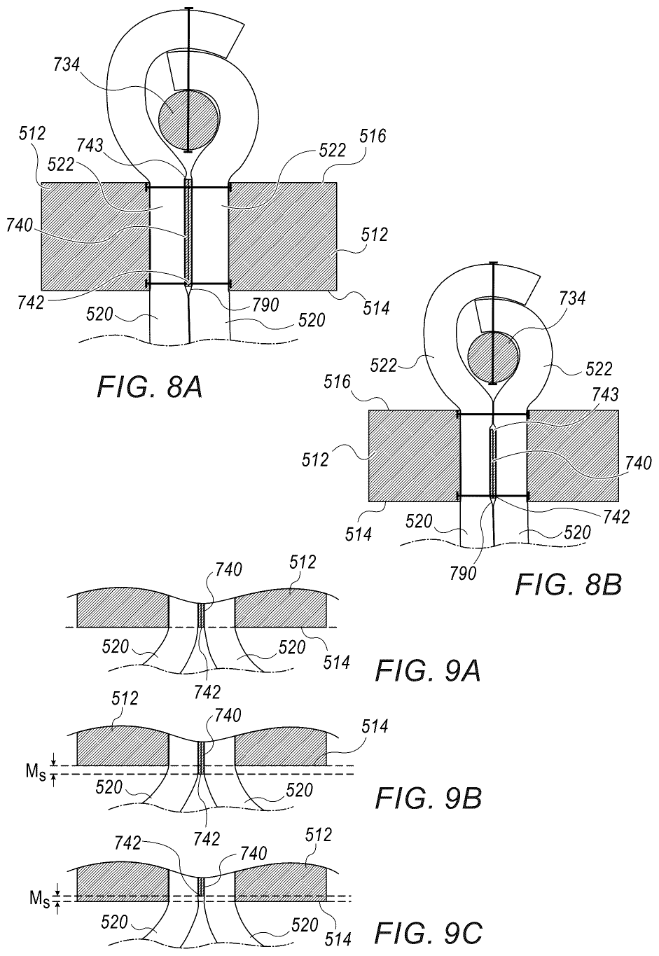

FIGS. 10A-10B show a partial top view and a partial perspective view of an exemplary commissure assembly 830 comprising a coupling member 838, and a separator 840 disposed between the leaflets 520. Separator 840 can be similar to any example described above with respect to separator 740, and in the interest of brevity will not be described further. Commissure assembly can be similar to commissure assembly 730, with the exception that it includes coupling member 838 disposed over the first face 524 of tabs 522.

Commissure assembly 830 is also shown to have the outermost portions of tabs 522, extending radially away from outer wedge 834 (which is also similar to any example of outer wedge 734) arranged flat with their second faces 526 facing each other. It is to be understood that this arrangement of the tabs 522 can be similarly applicable to commissure assemblies 730 described above with respect to FIGS. 7A-7B, instead of the tabs 522 wrapped over each other and over the outer wedge. Similarly, the commissure assembly 830 can be modified such that the tabs 522 are wrapped over each other and over outer wedge 834 in a similar manner to that illustrated in FIGS. 7A-7B Likewise, as mentioned with respect to commissure assembly 730, the arrangement of the tabs 522 is shown in FIGS. 8A-8B merely for illustrative purposes, and any other commissure assembly know in the art, comprising a coupling member 838, is applicable, modified to further include the separator 840.

FIGS. 8A-8B show coupling member 838 disposed around first face 524 of the tabs 522a, 522b, for example extending between the tabs 522 and the sidewalls 512 at the portions that pass through the commissure window 510, with free ends of the coupling member 838 extending from the commissure window inner surface 514. These free ends can be optionally wrapped around the sidewalls 512, back in the radially outward direction, for example. As described above for commissure assembly 730, any number of sutures 836 can be employed to join the tabs 522 to each other and/or to other components of the commissure assembly 830. For example, a suture 836a can be stitched through the tabs 522a, 522b and the coupling member 838 at a position radially outward from outer wedge 834. Additional sutures 836b, 836c can be employed to join tabs 522a, 522b, along with coupling member 838 and separator 840.

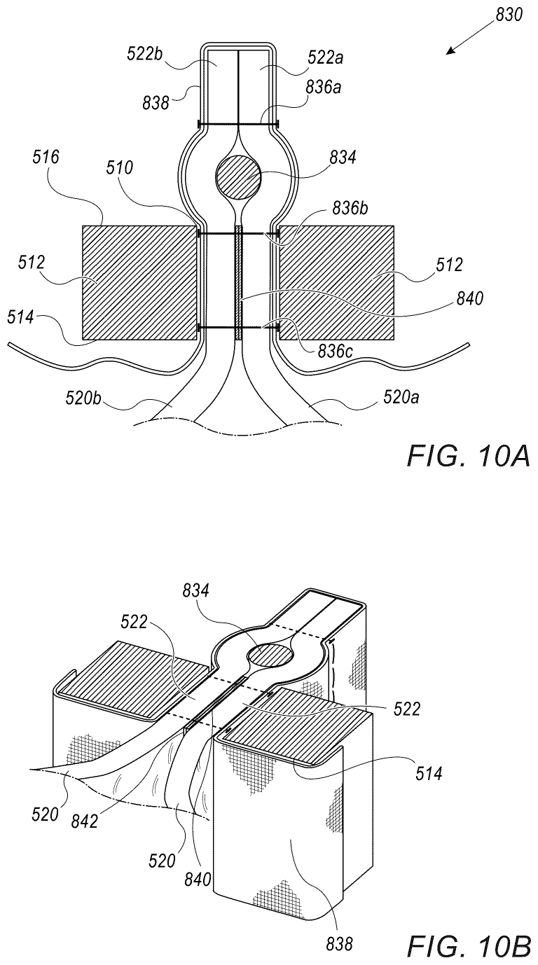

FIGS. 11A-11B show steps in the formation of another commissure assembly 930, in which a separator 940 is formed as an integral portion of a coupling member 938. FIG. 11A shows an initial step of forming the commissure assembly 930, for example prior to insertion of an outer wedge 934 and/or prior to mounting the commissure assembly 930 in commissure window 510. FIG. 11B shows the final configuration of commissure assembly 930, which can be similar to commissure assembly 830 in all respects, and can optionally include outer wedge 934 that can be similar to outer wedge 834, 734, except that commissure assembly 930 does not include a separate independent separator, but rather a coupling member 938 can be configured to have a portion thereof extending between both leaflets 520, effectively serving as a separator 940.

Coupling member 938 can be similar to coupling member 838 described above with respect to FIGS. 10A-10B, except that it does not only cover the first face 524 of tabs 522a, 522b, but is also folded over itself between the tabs 522, as shown in FIG. 11A. Thus, the folded inner portion of the coupling member 938 disposed between the leaflets 520, and more specifically, between leaflet tabs 522, is a separator 940, wherein the separator inner end 942 is defined by the innermost fold of the coupling member 938 over itself. As is the case with any of the other commissure assemblies described above, any number of sutures 936 can be employed to join the tabs 522 to each other and/or to other components of the commissure assembly 930. For example, a suture 936a can be stitched through the tabs 522a, 522b and the coupling member 838 at a position radially outward from outer wedge 834, and a suture 936d can be stitched through both tabs 522, the layers of the coupling member 938, and the outer wedge 934. Additional sutures 936b, 936c can be employed to join tabs 522a, 522b, along with coupling member 938 and the portion of the coupling member 938 that serves as separator 940.

The coupling member 938 has a coupling member thickness Tc, such that the separator thickness Ts can be defined as twice the thickness of the coupling member 938 (i.e., Ts=2Tc). The separator thickness Ts can assume any value within the range of values described hereinabove with respect to separator 740. Thus, for a separator 940 to have a separator thickness Ts of about 300μ, the coupling member 938 can be provided with a thickness Tc of about 150μ. The coupling member 938 can comprise a thromboresistant material, for example by being made of TPU, or being provided as a fabric or cloth material coated with TPU. The separator inner end 942 can be positioned within a range of a distance Ms in any radial direction with respect to the commissure window inner surface 514, having a value not greater than Ts, as described above for separator 740 with respect to FIGS. 9A-9C.

Some commissure assembly configurations include folds of the leaflets, and particularly, of the tabs, along regions adjacent the frame, such as sidewalls of the commissure assembly, which are sutured in a manner that may result in high stress zones, occasionally causing such regions of the tabs to tear under repetitive loading.

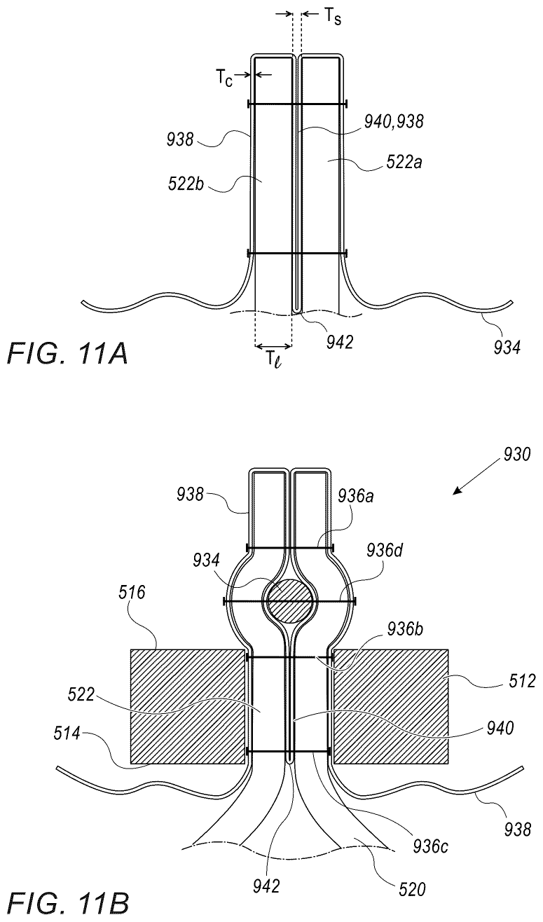

FIG. 12A shows a partial perspective view of a commissure assembly 1030 provided with a T-shaped separator 1040, arranged to couple the tabs 522 to each other and to the commissure window 510 in a manner designed to offset the stress zones from the folded portions of the tabs 522, so as to mitigate the risk of tear formation along the tabs over time. A T-shaped separator 1040 can include a radial extension 1046 extending in a radial direction and disposed between the tabs 522a, 522b at the portions extending through the commissure window 510, and a lateral head 1044 disposed in the circumferential direction, perpendicular to the radial direction and to the radial extension 1046.

In some examples, the T-shaped separator 1040 is made from a rigid material that can be coated by a thromboresistant material, such as TPU. The radial extension 1046 defines a separator inner end 1042, which can be positioned within a range of a distance Ms in any radial direction with respect to the commissure window inner surface 514, having a value not greater than Ts, as described above for separator 740 with respect to FIGS. 9A-9C. The separator thickness Ts is defined as the thickness of the radial extension 1046, and can have a value in any of the ranges described hereinabove with respect to separator thickness Ts of separator 740.

The lateral head 1044 is positioned radially away from the commissure window outer surface 516, wherein the tabs 522 can be wrapped over it such that the total lateral dimension of the lateral head 1004, and twice the thickness of the leaflets T1, exceeds the opening width Wc. Thus, the lateral head 1044 of a T-shaped separator 1040 can replace an outer wedge, thereby reducing the total amount of components utilized to form a commissure assembly 1030.

As shown, one tab 522a comprises a first tab portion 1050a extending through the commissure window 510, positioned between the sidewall 512a and the radial extension 1046. The tab 522a is then folded at a first fold 1058a, from which a second tab portion 1052a extends laterally or circumferentially away from radial extension 1046, positioned between lateral head 1044 and the outer surface 516 of sidewall 512a. Tab 522a is then folded again at second fold 1060a and extends radially outward across the edge of the lateral head 1044, and is then folded again at third fold 1062a to extend in a lateral or circumferential direction, back over an outer surface of lateral head 1044, terminating at tab edge 1048a.

The other tab 522b comprises a tab portion 1050b extending through the commissure window 510, positioned between the sidewall 512b and the radial extension 1046. The tab 522b is then folded at a second fold 1058b, from which a second tab portion 1052b extends laterally or circumferentially away from radial extension 1046, positioned between lateral head 1044 and the outer surface 516 of sidewall 512b. Tab 522b is then folded again at second fold 1060b and extends radially outward across the edge of the lateral head 1044, and is then folded again at third fold 1062b to extend in a lateral or circumferential direction, partially overlapping the first face 524 of fourth tab portion 1056a, terminating at tab edge 1048b.

A first suture 1064 forms an overcast stitch pattern 1066 along each fourth tab portion 1056, comprising an in-and-out stitch line 1068 distanced from the third fold 1062, and a series of bights 1070 extending from the in-and-out stitch line 1068 and looped over the tab edge 1048. For example, first suture 1064a forms an overcast stitch pattern 1066a along fourth tab portion 1056a, comprising an in-and-out stitch line 1068a passing vertically along the height of fourth tab portion 1056a, in parallel to and at a distance from third fold 1062a, and a series of bights 1070a, each forming a loop extending from a penetration point of the in-and-out stitch line 1068a into fourth tab portion 1056a, toward and around tab edge 1048a. Similarly, first suture 1064b forms an overcast stitch pattern 1066b along fourth tab portion 1056b, comprising an in-and-out stitch line 1068b passing vertically along the height of fourth tab portion 1056b, in parallel to and at a distance from third fold 1062b, and a series of bights 1070b, each forming a loop extending from a penetration point of the in-and-out stitch line 1068b into fourth tab portion 1056b, toward and around tab edge 1048b.

A second suture 1072 is passed in a zig-zag pattern between stitches of the in-and-out stitch line 1068 of the first suture 1064 of one tab, and the bights 1070 of the first suture 1064 of the opposite tab. For example, second suture 1072 is shown in FIG. 12A to comprise first wrap 1074 wrapped around a stitch of in-and-out stitch line 1068a, the suture 1072 extending from first wrap 1074 toward tab edge 1048b of the opposite tab 522b, at which point it forms a second wrap 1076 around a corresponding bight 1070b, extending therefrom back toward a subsequent stitch of the in-and-out stitch line 1068a to form another first wrap 1074, thus forming a continuous zig-zagged stitch pattern that serves to couple the fourth tab portions 1056 of both tabs 522a, 522b to each other, over the lateral head 1044.

In some examples, a third suture 1080 is added to further couple each tab 522 to a corresponding sidewall 512, each third suture 1080 comprises a series of sidewall loops 1082 looped around the corresponding sidewall 512, and a series of intermediary wraps 1084 extending between adjacent sidewall loops 1082, wrapped around the corresponding in-and-out stitch line 1068 of the corresponding first suture 1064.

FIG. 12A illustrates a third suture 1080a extending over a lateral outer side of sidewall 512a and over third tab portion 1054a toward in-and-out stitch line 1068a. The third suture 1080a is then passed between one stitch of the in-and-out stitch line 1068a and the fourth tab portion 1056, passing across a penetration point of the stitches toward and back beneath the subsequent stich of the same in-and-out stitch line 1068a, extending therefrom back over the third tab portion 1054 toward sidewall 512a, thereby forming intermediate wrap 1084a. The third suture 1080a extends between the second tab portion 1052a and the outer surface 516 of sidewall 512a, then radially inward between the first tab portion 1050a and sidewall 512a, and then laterally over the inner surface 514 of sidewall 512a, thereby forming sidewall loop 1082a, at which point subsequent intermediary wraps 1084 and sidewall loops 1082 can be similarly formed. While hidden from view in FIG. 12A, it is to be understood that a third suture 1080b can follow a similar pattern to couple tab 522b to sidewall 512b.

Advantageously, the commissure assembly 1030 described above and illustrated in FIG. 12A can efficiently couple both tabs 522 to each other, as well as to the sidewalls 512 of commissure window 510, wherein the single line of penetration points into the tabs 522 is along the in-and-out stitch lines 1068 which are distanced away from the fold (e.g., from third folds 1062). All other sutures do not penetrate into the tissue but are rather wrapped over portions of the in-and-out stitch lines 1068, such that stress concentrations at the penetration points are offset away from leaflet and tab folds, and away from regions that may be prone to elevated stresses under repetitive loading, thereby reducing the risk of tear formation and improving long-term durability.

FIG. 12B shows an exemplary commissure assembly 1130, which is a variation of commissure assembly 1030, similar thereto in all respects, except that the T-shaped separator 1140 can be formed from a plate which is bent over itself to form the radial extension 1146. The manner by which the tabs 522 are folded and sutured to form commissure assembly 1130 is similar to any of the examples described above with respect to commissure assembly 1030, with like reference numerals referring to like components, and in the interest of brevity will not be described further.

A deformable rigid plate, such as a metallic plate, can be provided with a plate thickness Tp. As shown in FIG. 12B, the separator thickness Ts, defined as the thickness of the radial extension 1146, is equal to at least twice the plate thickness Tp. Ts can be equal to 2Tp is both plate portions are tightly pressed against each other when forming the radial extension 1146, or can be somewhat greater than 2Tp if they are slightly spaced apart from each other. The rigid plate can be formed from a shape-memory (e.g., Nitinol) or a spring-like material, which tends to bias both portions forming the radial extension 1146 away from each other (i.e., toward sidewall 512). This configuration may be, in some instances, advantageous over a single-unit T-shaped separator 1040 illustrated in FIG. 12A, as the portions of the plate forming radial extension 1146 in such implementations can further press the tabs 522, and more specifically, first tab portions 1150, tightly against the corresponding sidewalls 512, thereby improving their grip against the commissure window 510.

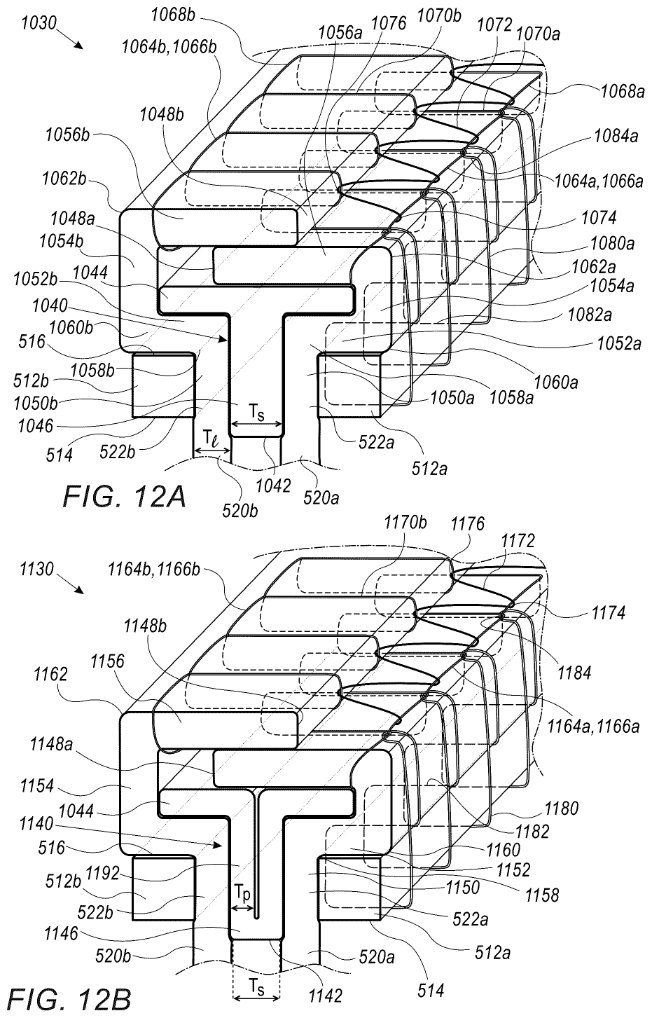

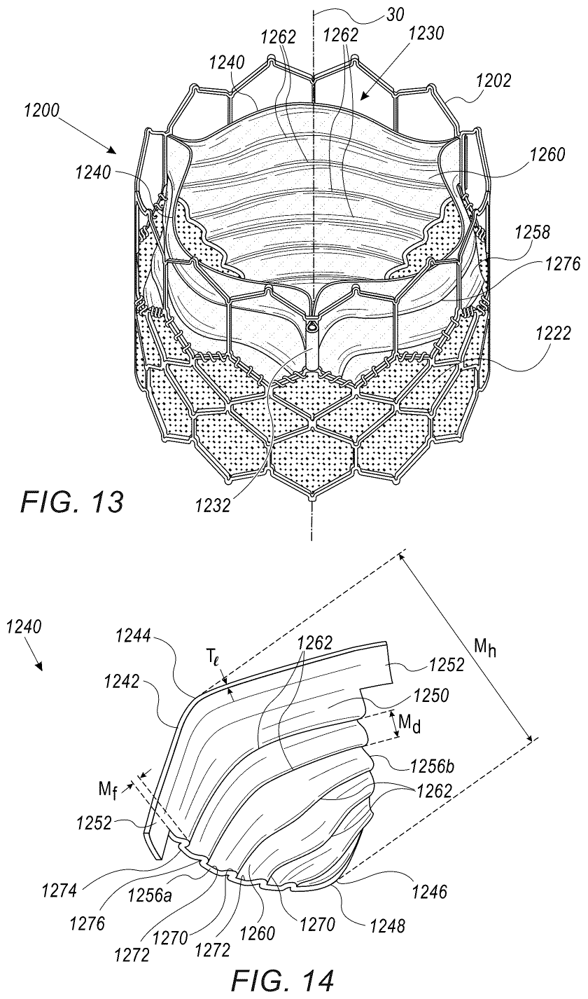

FIG. 13 illustrates a high-level perspective view of a prosthetic valve 1200, in accordance with some examples. Prosthetic valve 1200 is similar to any of the prosthetic valves 100, 300 or 400, with like reference numerals referring to like components thereof, and may be implemented according to any of the examples and optional configurations described above for prosthetic valve 100, 300, 400 except that it includes leaflet assembly 1230 wherein each leaflet 1240 includes a plurality of lateral folds 1262 extending between the leaflet body sidelines 1256a and 1256b, as also shown in greater detail in FIG. 14. In particular, prosthetic valve 1200 comprises: a frame 1202 having an inflow end 1204 and an outflow end 1206; with the leaflet assembly 1230 mounted within frame 1202. Three leaflets 1240 are illustrated, however this is not meant to be limiting in any way. Particularly, while three leaflets 1240 arranged to collapse in a tricuspid arrangement similar to the native aortic valve are illustrated, it will be clear that prosthetic valve 1200 can include any other number of leaflets 1240, such as two leaflets configured to collapse in a bicuspid arrangement similar to the native mitral valve, or more than three leaflets, depending upon the particular application.

As mentioned above, the term “prosthetic valve” refers to any type of a prosthetic valve deliverable to a patient's cardiac target site, which is radially expandable and compressible between a radially compressed, or crimped, state, and a radially expanded state. In one example, a prosthetic valve constructed differently than prosthetic valve 1200 can be provided, without exceeding the scope of the disclosure. In one example, prosthetic valve 1200 is constructed so as to be mounted within the native aortic valve, the native mitral valve, the native pulmonary valve and/or the native tricuspid.

In one example, as also illustrated in FIG. 13, prosthetic valve 1200 further comprises: an inner skirt 1222 and a plurality of commissure assemblies 1232. In another example (not shown), prosthetic valve 1200 further comprises an outer skirt. In one example, frame 1202 comprises a plurality of interconnected struts 1212 that can be constructed according to any of the examples described hereinabove with respect to struts 112, and can include angled struts 1214, vertical struts 1216 that can comprise both outflow vertical struts and inflow vertical struts, wherein at least some of the outflow vertical struts can include commissure windows. With further similarity to any example described above for struts 112, struts 1212 can intersect at junctions 1220.

Frame 1202, as well as inner skirt 1222 and the outer skirt, can be similar in structure and function to any example described hereinabove with respect to frame 102, and in the interest of brevity will not be described further.

In one example, each adjacent pair of leaflets 1240 are secured to each other at a respective commissure assembly 1232. The commissure assemblies 1232 can be similar in structure and function to any example described herein above with respect to commissure assembly 132 or any other configuration of commissure assemblies disclosed herein or known in the art, and may be similarly utilized to mount the leaflet assembly 1230 to the frame 1202 such that the leaflet assembly 1230 is attached to the frame 1202 at the outflow portions via commissure assemblies 1232, and along the cusp edges 1246 along a scallop line that can be optionally sutured to the inner skirt 1222.

In one example, leaflets 1240 and tabs 1252 are formed of a continuous piece of material. In another example, separate leaflets 1240 are provided, and are separately mounted within frame 1202 and/or secured to each other.

Although prosthetic valve 1200 is illustrated and described herein as comprising an inner skirt 1222, this is not meant to be limiting in any way. In another example (not shown), inner skirt 1222 is not provided. In another example, an outer skirt is not provided. In one example, inner skirt 1222 is arranged to function as a sealing member to prevent or decrease perivalvular leakage.

In one example, leaflets 1240 are secured to inner skirt 1222 such that inner skirt 1222 functions as an anchoring region for anchoring leaflets 1240 to frame 1202 and/or functions to protect leaflets 1240 against damage which may be caused by contact with frame 1202, for example during valve crimping or during working cycles of prosthetic valve 300. In one example, the cusp edge 1246 of leaflets 1240 secured to inner skirt 1222 exhibits an undulating, curved and/or scalloped shape.

In one example, where an outer skirt is provided (not shown), the outer skirt is arranged to function as a sealing member retained between frame 1202 and the surrounding tissue of the native annulus against which prosthetic valve 1200 is mounted, thereby reducing risk of paravalvular leakage past prosthetic valve 1200.

FIG. 14 illustrates a high-level perspective view of one example of a leaflet 1240, which can be similar to any of the leaflets 140 or 240, with like reference numerals referring to like components thereof, except that it includes a plurality of lateral folds 1262. In particular, each leaflet 1240 has a free edge 1242 defining a free edge midpoint 1244, a rounded cusp edge 1246 defining a cusp edge midpoint 1248 opposing free edge midpoint 1244, and a pair of generally oppositely directed tabs 1252 separating the cusp edge 1246 and the free edge 1242. The leaflet body 1250 extends between the cusp edge 1246 and the free edge 1242, and defines two leaflet body sidelines 1256a and 1256b, extending between the cusp edge midpoint 1248 and the free edge 1242. The leaflet 1240 defines a leaflet first face 1258, that can be also referred to as a leaflet outer face, facing away from the central axis 30 of the valve when the leaflet assembly 1230 is mounted in the frame 1202, and a leaflet second face 1260, that can be also referred to as a leaflet inner face, facing toward the central axis 30 and toward the other leaflets 1240 of the leaflet assembly 1230 when mounted in the frame.

A plurality of lateral wrinkles or folds 1262 are spaced from each other between the free edge 1242 and a the cusp edge midpoint 1248. Each lateral fold 1262 extends between the leaflet body sidelines 1256a and 1256b, protruding radially inward, toward the main axis 30 and toward the other leaflets 1240 of the leaflet assembly 1230, when mounted in the frame 1202. Unlike any of lateral fold 162 of leaflet 140 or bulge 262 of leaflet 260, the lateral folds 1262 are not formed solely in response to flow through prosthetic valve 1200, but are rather created in a free state of the leaflets, even prior to attachment of the leaflet assembly 1230 to the frame 1202. Such lateral winkles or folds 1262 can advantageously improve robustness of the leaflet assembly 1230 within a prosthetic valve 1200, exploiting the wrinkled or folded regions for improving co-aptation during the diastolic phases, without increasing pressure gradients across the valve 1200 during the systolic phases.

The number of lateral folds 1262 of each leaflet 1240 can vary, as well as the distance between subsequent lateral folds 1262. In some examples, each leaflet 1240 includes at least 3 lateral folds. In some examples, each leaflet 1240 includes at least 5 lateral folds. In some examples, each leaflet 1240 includes at least 8 lateral folds. In some examples, each leaflet 1240 includes at least 10 lateral folds.

FIG. 14 illustrates a plurality of lateral folds 1262 defining, along leaflet second face 1260, a corresponding plurality of inner peaks 1270 protruding radially inward (toward main axis 30) and inner valleys 1272 formed between subsequent folds. Inner peaks 1270 and valleys 1270 are formed on leaflet second face 1260, while the leaflet first face 1258 can include a corresponding plurality of outer peaks 1274, closer to the frame 1202, and outer valleys 1276, farther from the frame 1202. If the leaflet 1240 has a substantially uniform thickness Tl, then the leaflet first face 1258 and second face 1260 are similarly shaped, such that the outer valleys 1276 of leaflet first face 1258 are formed opposite to the inner peaks 1270 of leaflet second face 1260 across the leaflet's thickness, and outer peaks 1274 of leaflet first face 1258 are formed opposite to the inner valleys 1272 of leaflet second face 1260 across the leaflet's thickness. The inner valleys 1272 are generally defined at the base of the folds 1262 along leaflet second face 1260, for example between adjacent lateral folds, and can be generally representative of the level of leaflet second face 1260 if the leaflet would not include any folds (i.e., at the level of leaflet second face 160 for an alternative leaflet 140 provided with similar dimensions and shape as that of leaflet 1240, but without lateral folds).

Fold depth Mf designates the height or distance between the inner valleys 1272 and the corresponding inner peaks 1270 of a lateral fold 1262. The fold depth Mf of each lateral fold 1262 can vary along the length of the fold 1262 between the leaflet body sidelines 1256a and 1256b, and can have lower values closer to the leaflet body sidelines 1256a and 1256b. A maximal fold depth Mf of any single lateral leaflet 1262 is defined as the greatest value of Mf (i.e., the maximal fold) along the length of the corresponding fold 1262 between the leaflet body sidelines 1256.

Different lateral folds 1262 of the same leaflet 1240 can have different maximal fold depths Mf. However, each lateral fold 1262 of a plurality of folds 1262 of a leaflet 1240 will have a maximal fold depth Mf that is greater than the thickness Tl of the same leaflet 1240. In some examples, the maximal fold depth Mf of each lateral fold 1262 is greater than twice the leaflet thickness Tl of the leaflet. In some examples, the maximal fold depth Mf of each lateral fold 1262 is at least three times as great as the leaflet thickness Tl. In some examples, the maximal fold depth Mf of each lateral fold 1262 is at least five times as great as the leaflet thickness Tl. In some examples, the maximal fold depth Mf of each lateral fold 1262 is at least eight times as great as the leaflet thickness Tl. In some examples, the maximal fold depth Mf of each lateral fold 1262 is at least ten times as great as the leaflet thickness Tl.

Lateral folds are spaced from each other between the cusp edge midpoint 1248 and the free edge 1242, wherein fold-spacing Md indicated in FIG. 14, designates the distance between two adjacent lateral folds 1262, which can be measured, for example, between the inner peaks 1270 of the corresponding lateral folds 1262, and is significantly greater than the leaflet thickness Tl. In some cases, some of the lateral folds 1262 may not be completely parallel to each other between the leaflet body sidelines 1256, such that the distance between inner peaks 1270 of adjacent lateral folds 1262 can vary to some extent, depending on the proximity to any of the leaflet body sidelines 1256. In such cases, the term “fold-spacing Md” refers to the greatest distance between inner peaks 1270 of corresponding adjacent lateral folds 1262 measured along their lateral path, between lateral body sidelines 1256a and 1256b.

Since a leaflet 1240 can include more than two lateral folds 1262, the fold-spacing Md of each couple of adjacent lateral folds 1262 may be different. The term “minimal fold-spacing Md” refers to the lowest of Md values of all couples of adjacent lateral folds 1262 of a leaflet 1240. The term “maximal fold-spacing Md” refers to the highest of Md values of all couples of adjacent lateral folds 1262 of a leaflet 1240. Both minimal and maximal Md values can affect the behavior of the leaflet, responsive to the blood flow within the patient's body. In some examples, the minimal fold-spacing Md is at least three times as great as the leaflet thickness Tl. In some examples, the minimal fold-spacing Md is at least five times as great as the leaflet thickness Tl. In some examples, the minimal fold-spacing Md is at least eight times as great as the leaflet thickness Tl. In some examples, the minimal fold-spacing Md is at least ten times as great as the leaflet thickness Tl. In some examples, the maximal fold-spacing Md is at least three times as great as the leaflet thickness Tl. In some examples, the maximal fold-spacing Md is at least five times as great as the leaflet thickness Tl. In some examples, the maximal fold-spacing Md is at least eight times as great as the leaflet thickness Tl. In some examples, the maximal fold-spacing Md is at least ten times as great as the leaflet thickness Tl.

When the prosthetic valve is implanted in a patient's body, the shape of the lateral folds, including their fold depths Mf and fold-spacings Md, may vary in response to blood flow and pressure varying between diastolic and systolic phases. Thus, it is to be understood that any references to relative values of fold depths Mf and/or to fold-spacing Md, including any of maximal fold depths, minimal fold-spacings, and/or maximal fold-spacings, refer to such values in a free state of the leaflets 1240, for example as measured when the leaflet assembly 1240 is mounted in the frame 1202, in an expanded state of the prosthetic valve, while the valve 1200 is outside of a patient's body and is not subjected to any external forces.

Leaflet body height Mh designates the linear distance between the cusp edge midpoint 1248 and the free edge midpoint 1244. Leaflet body height Mh can vary between a low value in a free state of the leaflet 1240, shown for example in FIG. 13, and a greater value in a stretched state of the leaflet. The leaflet 1240 is termed to be in a free state when it is not stretched between its cusp edge 1246 and free edge 1242. For example, the leaflets 1240 can be in a free state when they are biased radially outward, toward the frame 1202, as shown in FIG. 13. This can be, for example, during systole (for aortic replacement valves), during blood flow through the prosthetic valve 1200.

It is to be understood that a leaflet 1240 that includes a plurality of lateral folds 1262 can be either a flattenable leaflet or a non-flattenable leaflet. A non-flattenable leaflet 1230 can be implemented according to any example described above with respect to non-flattenable leaflet 240, with the inclusion of a series of lateral folds 1262 as described hereinabove.

Various methods can be implemented to form a leaflet 1240 with a plurality of lateral folds 1262. In one example, the leaflet material can be gathered at the leaflet body sidelines 1256. The folds at the leaflet body sidelines 1256 can be joined by seams or other sutures (not shown), resulting in a series of pleat-like lateral folds 1262 along the leaflet body 1250.

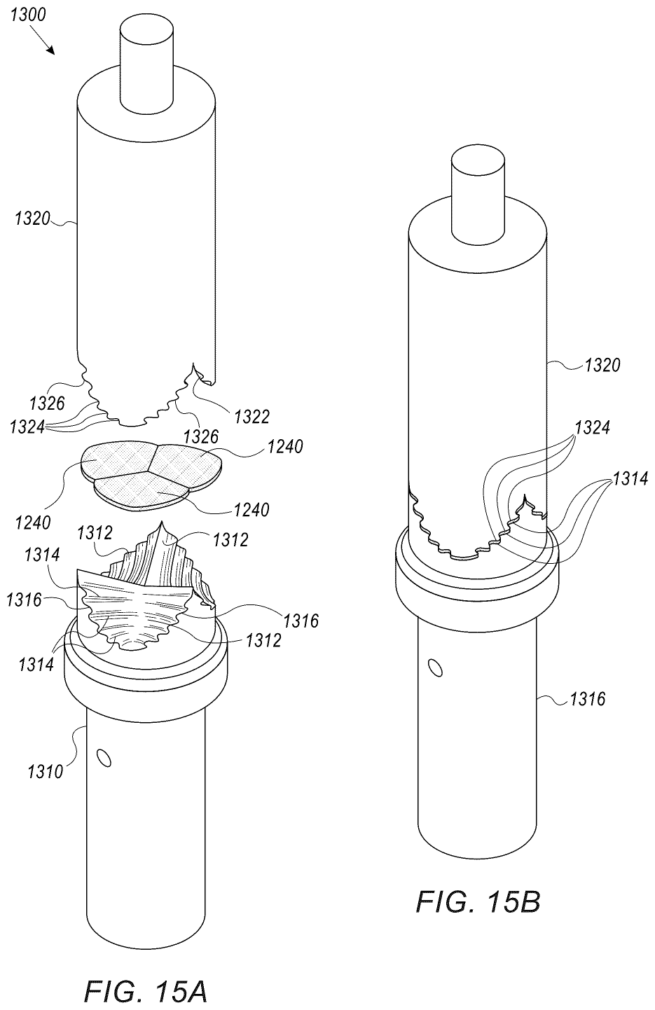

FIGS. 15A-15B illustrate another example for forming leaflets 1240 with lateral folds 1262 by forging. FIG. 15A shows a two-part leaflet shape forming apparatus 1300 that includes a base portion 1310 and a die portion 1320. The base portion 1310 can include a mandrel that defines one or more concave surfaces 1312, such as the three concave surfaces 1312 of the illustrated example. The concavity of each surface 1312 is designed to match the desired concavity of leaflet first face 1258. Each concave surface 1312 further includes a series of protrusions 1314 extending laterally between edges 1316 thereof, shaped and dimensioned to match the desired lateral folds 1262. The die portion 1320 defines one or more convex surfaces 1322, matching in number to the number of concave surfaces 1312, and having a shape complementary to that of the concave surfaces 1312. Particularly, each convex surface 1322 includes a series of recesses 1324 extending laterally between edges 1326, complementary to the matching protrusions 1314.

As shown in FIG. 15A, leaflets 1240, which can be provided in a flattened and relatively smooth configuration, are placed over the concave surfaces 1312, and are then pressed between the concave surfaces 1312 of the base portion 1310 and the convex surface 1322 of the die portion 1320, to assume the final shape with the plurality of lateral folds 1262.

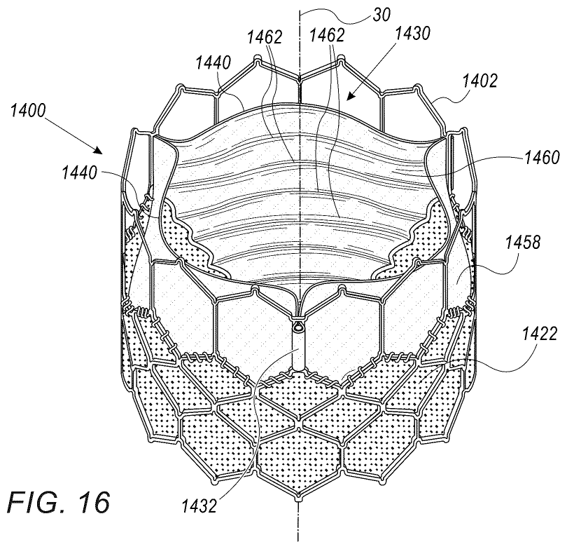

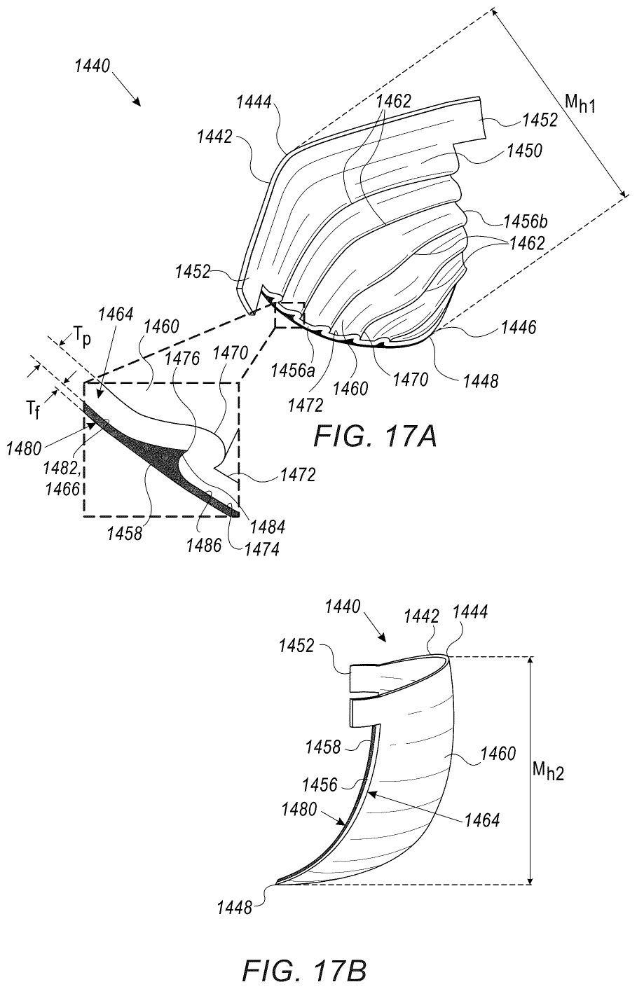

FIG. 16 illustrates a high-level perspective view of a prosthetic valve 1400, in accordance with some examples. Prosthetic valve 1400 is identical to prosthetic valve 1200, with like reference numerals referring to like components thereof, except that the leaflet assembly 1430 include leaflets 1440, shown in greater detail in FIGS. 17A-B, each formed of two layers: an inner layer 1464 and an outer layer 1480, the inner layer provided with a plurality of lateral folds 1462 having inner peaks 1470 extending radially inward (i.e., toward central axis 30) along leaflet second face 1460, while the leaflet first or outer face 1458, facing the frame 1402, is relatively smooth and devoid of peaks or valleys.

FIG. 17A illustrates a high-level perspective view of one example of a leaflet 1440, with a zoomed-in view of a portion of the leaflet. Leaflet 1440 can be similar to leaflet 1240, with like reference numerals referring to like components thereof, except that it further includes an additional outer layer 1480 facing the frame 1402 when assembled in prosthetic valve 1400, In particular, each leaflet 1440 has a free edge 1442 defining a free edge midpoint 1444, a rounded cusp edge 1446 defining a cusp edge midpoint 1448 opposing free edge midpoint 1444, and a pair of generally oppositely directed tabs 1452 separating the cusp edge 1446 and the free edge 1442. The leaflet body 1450 extends between the cusp edge 1446 and the free edge 1442, and defines two leaflet body sidelines 1456a and 1456b, extending between the cusp edge midpoint 1448 and the free edge 1442. The leaflet 1440 defines a leaflet first face 1458, that can be also referred to as a leaflet outer face, facing away from the central axis 30 and toward the frame 1402 when the leaflet assembly 1430 is mounted in the prosthetic valve, and a leaflet second face 1460, that can be also referred to as a leaflet inner face, facing toward the central axis 30 and toward the other leaflets 1440 of the leaflet assembly 1430 when mounted in the prosthetic valve.

Leaflet 1440 includes two layers: an inner layer 1464 that can be generally similar to the leaflet 1240 described above with respect to FIGS. 13 and 14, and an outer layer 1480 attached thereto. The inner layer 1464 defines the leaflet second or inner face 1460, facing the central axis 30 and toward the other leaflets 1440 of the leaflet assembly 1430 when mounted in the prosthetic valve, and an inner layer first face 1466, opposite to the leaflet second face 1460. The outer layer 1480 defines the leaflet first or outer face 1458, facing the frame 1402 when mounted in the prosthetic valve, and an outer layer second face 1482, opposite to the leaflet outer face 1458. As shown in the zoomed-in view of FIG. 17A, the outer layer second face 1482 is attached to the inner layer first face 1466.

Inner layer 1464 can be formed according to any method described above for leaflet 1240, including gathering along the sidelines or utilizing leaflet shape-forming apparatus 1300. Specifically, the inner layer includes a plurality of lateral wrinkles or folds 1462 are spaced from each other between the free edge 1442 and a the cusp edge midpoint 1448. Each lateral fold 1462 extends between the leaflet body sidelines 1456a and 1456b, protruding radially inward, toward the main axis 30 and toward the other leaflets 1440 of the leaflet assembly 1430, when mounted in the prosthetic valve Similar to folds 1262, the lateral folds 1462 are not formed solely in response to flow through prosthetic valve 1400, but are rather created in a free state of the leaflets, even prior to attachment of the leaflet assembly 1430 to the frame 1402.

The number of lateral folds 1462 of each leaflet 1440 can vary, as well as the distance between subsequent lateral folds 1462. In some examples, each leaflet 1440 includes at least 3 lateral folds. In some examples, each leaflet 1440 includes at least 5 lateral folds. In some examples, each leaflet 1440 includes at least 8 lateral folds. In some examples, each leaflet 1440 includes at least 10 lateral folds.

FIG. 17A illustrates a plurality of lateral folds 1462 defining, along leaflet second face 1460, a corresponding plurality of inner peaks 1470 protruding radially inward (toward main axis 30) and inner valleys 1472 formed between subsequent folds. Inner peaks 1470 and valleys 1470 are formed along the inner layer 1464, on leaflet second face 1460, while the inner layer first face 1466 can include a corresponding plurality of inner layer outer peaks 1474 and inner layer outer valleys 1476. The inner layer 1464 can have a substantially uniform thickness Tp, such that the inner layer first face 1466 and leaflet second face 1460 are similarly shaped, wherein the inner layer outer valleys 1476 are formed opposite to the inner peaks 1470 of leaflet second face 1460 across the inner layer's thickness, and inner layer outer peaks 1474 are formed opposite to the inner valleys 1472 of leaflet second face 1460 across the inner layer's thickness. The inner valleys 1472 are generally defined at the base of the folds 1462 along leaflet second face 1460, for example between adjacent lateral folds, and can be generally representative of the level of leaflet second face 1460 if the leaflet would not include any folds (i.e., at the level of leaflet second face 160 for an alternative leaflet 140 provided with similar dimensions and shape as that of leaflet 1440, but without lateral folds).

Fold depth Mf, including maximal fold depth, can be defined for lateral folds 1462 in the same manner described above for lateral folds 1262 of leaflet 1240, with the exception that maximal fold depth Mf of folds 1462 can be measured as a function of inner layer thickness Tp instead of leaflet thickness Tl. Thus, each lateral fold 1462 of a plurality of folds 1462 of an inner layer 1464 will have a maximal fold depth Mf that is greater than the thickness Tp of the same inner layer 1464. In some examples, the maximal fold depth Mf of each lateral fold 1462 is greater than twice the inner layer thickness Tp. In some examples, the maximal fold depth Mf of each lateral fold 1462 is at least three times as great as the inner layer thickness Tp. In some examples, the maximal fold depth Mf of each lateral fold 1462 is at least five times as great as the inner layer thickness Tp. In some examples, the maximal fold depth Mf of each lateral fold 1462 is at least eight times as great as the inner layer thickness Tp. In some examples, the maximal fold depth Mf of each lateral fold 1462 is at least ten times as great as the inner layer thickness Tp.

Fold-spacing Md, including minimal fold-spacing and maximal fold-spacing, can be defined for lateral folds 1462 in the same manner described above for lateral folds 1262 of leaflet 1240, with the exception that minimal fold-spacing Md between folds 1462 can be measured as a function of inner layer thickness Tp instead of leaflet thickness Tl. Thus, fold-spacing Md, which can be measured, for example, between the inner peaks 1470 of adjacent lateral folds 1462, is significantly greater than the inner layer thickness Tp. In some examples, the minimal fold-spacing Md is at least three times as great as the inner layer thickness Tp. In some examples, the minimal fold-spacing Md is at least five times as great as the inner layer thickness Tp. In some examples, the minimal fold-spacing Md is at least eight times as great as the inner layer thickness Tp. In some examples, the minimal fold-spacing Md is at least ten times as great as the inner layer thickness Tp. In some examples, the maximal fold-spacing Md is at least three times as great as the inner layer thickness Tp. In some examples, the maximal fold-spacing Md is at least five times as great as the inner layer thickness Tp. In some examples, the maximal fold-spacing Md is at least eight times as great as the inner layer thickness Tp. In some examples, the maximal fold-spacing Md is at least ten times as great as the inner layer thickness Tp.

Outer layer 1480 is made of an elastic and compressible material. A biocompatible elastic an compressible material can include natural tissue, such as a tissue comprising elastin fibers, or a synthetic material, including polymers, sponge-like materials, and the like. The outer layer 1480 defines a relatively smooth leaflet first face 1458, meaning that it does not include peaks and valleys of the type defined by lateral folds 1462, and more specifically, meaning that even if small peaks are formed along the leaflet first face 1458, their fold depth is significantly smaller than the fold depth Mf of lateral folds 1462, and preferably smaller than half the fold depth Mf of lateral folds 1462.

The outer layer 1480 is attached to the inner layer 1464 such that it fills the volume between the relatively smooth leaflet first face 1458 and inner layer outer valleys 1476. Thus, the outer layer second face 1482, directly attached to the inner layer first face 1466, comprises a plurality of outer layer peaks 1482 and outer layer valleys 1486, such that the outer layer peaks 1484 extend into the corresponding inner layer valleys 1476, in a free state of the leaflet 1440. FIG. 17A shows a leaflet 1440 in a free state thereof, wherein a minimal outer layer thickness Tf is defined as the thickness of the outer layer 1480 between the leaflet first face 1458 and the outer layer valleys 1486, in the free state of leaflet 1440. Valve thickness Tl can be defined as the sum of minimal outer layer thickness Tf and inner layer thickness Tp. Notably, the outer layer 1480 can have varying thickness in the free state of the leaflet shown in FIG. 17A, ranging from the minimal outer layer thickness Tf to a maximal value at the region of outer layer peaks 1484, which can be the sum of minimal outer layer thickness Tf and the fold depth Mf. The outer layer 1480 can be coated over inner layer first face 1466, or otherwise bonded or attached thereto, such as by gluing (e.g., utilizing biological adhesives), tissue welding, and the like.

The minimal outer layer thickness Tf is preferably substantially smaller than the inner layer thickness Tp. In some examples, the inner layer thickness Tp is at least three times greater than the minimal outer layer thickness Tf. In some examples, the inner layer thickness Tp is at least five times greater than the minimal outer layer thickness Tf. In some examples, the inner layer thickness Tp is at least ten times greater than the minimal outer layer thickness Tf.

Leaflet body height Mh can be defined for lateral folds 1462 in the same manner described above for lateral folds 1262 of leaflet 1240, designating the linear distance between the cusp edge midpoint 1448 and the free edge midpoint 1444. FIG. 17A shows leaflet 1440 in a free state, and FIG. 17B shows the leaflet 1440 in a stretches state. As defined for leaflet 1240, leaflet 1440 is termed to be in a free state when it is not stretched between its cusp edge 1446 and free edge 1442. For example, the leaflets 1440 can be in a free state when they are biased radially outward, toward the frame 1402, as shown in FIG. 16. This can be, for example, during systole (for aortic replacement valves), during blood flow through the prosthetic valve 1400. A leaflet 1440 can assume a stretched state, stretched between its cusp edge 1446 and free edge 1442 as shown in FIG. 17B, when it co-apts with other leaflets of the leaflet assembly 1430, such as during diastole (for aortic replacement valves). During co-aptation, the leaflet 1440 is stretched in a manner that straightens to some degree at least some of the lateral folds 1462. This can cause the leaflet second face 1460 to assume a relatively smooth configuration, meaning that even if the lateral folds 1462 are not totally flattened, their fold depths Mf are smaller than in the free state.

As shown in FIGS. 17A and 17B, the leaflet body height Mh can assume a first leaflet body height Mh1 in a free or unstretched state of the leaflet, and elongate to a second leaflet body height Mh2 in a stretched state, wherein Mh2 is greater than Mh1 due to the straightening of lateral folds 1462. In the stretched state, as also shown in FIG. 17B, the second layer 1480 can assume a thinner, more homogenous thickness across the leaflet 1440. This can be achieved, for example, as a result of rearrangement of fibers (e.g., elastin fibers for a tissue material that can include such fibers) that can be rearranged accordingly.

Advantageously, lateral folds 1260 or 1460 of respective leaflets 1240 or 1440 can transition, between the free and stretches states of the leaflets, from a first configuration in which the lateral folds have fold depths Mf to second configuration, in which the lateral folds are straighter or flatter, can increase the leaflets' thromboresistance. Specifically, post-implantation biological repose can result in thrombosis of tissue overgrowth along the surfaces of the leaflets, which can pose a risk in case of thrombus detachment or interfere with leaflet motility over time. By forming the leaflets with a series of lateral folds that stretch in each transition of the leaflet, for example between systolic and diastolic phases, the frequent transition of the lateral folds between their naturally folded configuration and stretched or flattened configuration can prevent tissue overgrowth or adherence of pro-thrombotic factors thereto, thereby mitigating risks associated therewith.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising:
a frame having an inflow end and an outflow end, wherein the frame is movable between a radially compressed and a radially expanded state; and
a plurality of leaflets secured to the frame, each leaflet having: a leaflet first face and a leaflet second face opposing the leaflet first face, a free edge defining a free edge midpoint, and an opposing cusp edge, the cusp edge defining a cusp edge midpoint and first and second leaflet body sidelines, each leaflet body sideline extending from the cusp edge midpoint to the free edge,
wherein each of the plurality of leaflets is not flattenable.

Example 2. The prosthetic valve of any example herein, particularly example 1, wherein, for each of the plurality of leaflets, the leaflet first face and the leaflet second face are non-developable surfaces.

Example 3. The prosthetic valve of any example herein, particularly any one of examples 1 or 2, wherein, for each of the plurality of leaflets, the leaflet first face is concave.

Example 4. The prosthetic valve of any example herein, particularly example 3, wherein, for each of the plurality of leaflets, the concavity of the first face is three-dimensional.

Example 5. The prosthetic valve of any example herein, particularly any one of examples 1 to 4, wherein, for each of the plurality of leaflets, the leaflet second face is convex.

Example 6. The prosthetic valve of any example herein, particularly example 5, wherein, for each of the plurality of leaflets, the convexity of the second face is three-dimensional.

Example 7. The prosthetic valve of any example herein, particularly any one of examples 1 to 6, wherein the plurality of leaflets comprises three leaflets.

Example 8. The prosthetic valve of any example herein, particularly any one of examples 1 to 7, wherein, for each of the plurality of leaflets, the respective leaflet is curved from the first leaflet body sideline to the second leaflet body sideline.

Example 9. The prosthetic valve of any example herein, particularly example 8, wherein the curve of each of the plurality of leaflets from the respective first leaflet body sideline to the respective second leaflet body sideline forms a predetermined section of an ellipsoid.

Example 10. The prosthetic valve of any example herein, particularly any one of examples 8 or 9, wherein each of the first leaflet body sideline and the second leaflet body sideline is curved.

Example 11. The prosthetic valve of any example herein, particularly any one of examples 1 to 10, wherein each of the plurality of leaflets is curved from the free edge to the cusp edge midpoint.

Example 12. The prosthetic valve of any example herein, particularly example 11, wherein an arc length of any curve extending from the free edge to the cusp edge midpoint exhibits a positive correlation with a distance between the respective curve and the closer one of the first leaflet body sideline or the second leaflet body sideline.

Example 13. The prosthetic valve of any example herein, particularly any one of examples 11 or 12, wherein an arc length of a curve extending from the free edge midpoint to the cusp edge midpoint is greater than any other curve extending from the first edge to the cusp edge midpoint.

Example 14. The prosthetic valve of any example herein, particularly any one of examples 1 to 13, wherein the frame defines an enclosure, at least a portion of each of the plurality of leaflets situated within the enclosure.

Example 15. The prosthetic valve of any example herein, particularly example 14, wherein, responsive to flow through the enclosure, a bulge is formed in each of the plurality of leaflets, the bulge extending between the free edge midpoint and the cusp edge midpoint.

Example 16. The prosthetic valve of any example herein, particularly example 15, wherein, for each of the plurality of leaflets, the bulge is formed in the leaflet second face.

Example 17. The prosthetic valve of any example herein, particularly example 16, wherein, for each of the plurality of leaflets, the bulge forms a corresponding indent in the leaflet first face.

Example 18. The prosthetic valve of any example herein, particularly any one of examples 16 or 17, wherein, for each of the plurality of leaflets, the bulge exhibits a pair of opposing bulge sides, each bulge side defining a respective concave curve in the leaflet second face.

Example 19. The prosthetic valve of any example herein, particularly any one of examples 16 or 17, wherein, for each of the plurality of leaflets, the bulge exhibits a bulge first side and an opposite bulge second side, a distance between the bulge first side and the first leaflet body sideline being essentially equal to a distance between the bulge second side the second leaflet body sideline.

Example 20. The prosthetic valve of any example herein, particularly any one of examples 15 to 19, wherein, for each of the plurality of leaflets, the bulge is formed in a central portion of the leaflet.

Example 21. The prosthetic valve of any example herein, particularly any one of examples 15 to 20, wherein, for each of the plurality of leaflets, the bulge extends from a bulge outflow end to a bulge inflow end, a distance between the bulge outflow end and the free edge being greater than a distance between the bulge inflow end and the cusp edge midpoint.

Example 22. The prosthetic valve of any example herein, particularly any one of examples 15 to 20, wherein, for each of the plurality of leaflets, the bulge extends from a bulge outflow end to a bulge inflow end, the bulge inflow end reaching the cusp edge midpoint.

Example 23. The prosthetic valve of any example herein, particularly any one of examples 21 or 22, wherein, for each of the plurality of leaflets, a width of the bulge decreases from the bulge outflow end to the bulge inflow end.

Example 24. The prosthetic valve of any example herein, particularly any one of examples 15 to 23, wherein, for each of the plurality of leaflets, a width of the bulge is 1/9 to ⅓ of a maximal distance between the leaflet body sidelines.

Example 25. The prosthetic valve of any example herein, particularly any one of examples 15 to 24, wherein, for each of the plurality of leaflets, a height to width ratio of the bulge is at least 1:1 to 4:1.

Example 26. The prosthetic valve of any example herein, particularly any one of examples 15 to 25, wherein, for each of the plurality of leaflets, a surface of the bulge is essentially smooth.

Example 27. The prosthetic valve of any example herein, particularly any one of examples 1 to 26, wherein the plurality of leaflets are secured to each other at a plurality of commissure assemblies, each of the plurality of commissure assemblies extending from a commissure assembly outflow end to a commissure assembly inflow end,
wherein the plurality of commissure assemblies are secured to the frame, and
wherein a center of coaptation of the plurality of leaflets is located between the commissure assembly inflow ends and the inflow end of the frame.

Example 28. The prosthetic valve of any example herein, particularly example 27, wherein a commissure outflow plane is defined by the plurality of commissure assembly outflow ends,
wherein a commissure inflow plane is defined by the plurality of commissure assembly inflow ends,
wherein a cusp midpoint plane is defined by the plurality of cusp edge midpoints, and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 29. The prosthetic valve of any example herein, particularly example 27, wherein a cusp midpoint plane is defined by the plurality of cusp edge midpoints, and wherein a portion of each of the plurality of leaflets is positioned between the cusp midpoint plane and the inflow end of the frame.

Example 30. The prosthetic valve of any example herein, particularly example 29, wherein a commissure outflow plane is defined by the plurality of commissure assembly outflow ends,
wherein a commissure inflow plane is defined by the plurality of commissure assembly inflow ends, and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 31. The prosthetic valve of any example herein, particularly any one of examples 1 to 30, wherein the prosthetic valve has a nominal radius and a nominal height, and wherein, for each of the plurality of leaflets, an arc length of the free edge is at least 2.2 times greater than the nominal radius of the prosthetic valve.

Example 32. The prosthetic valve of any example herein, particularly example 31, wherein, for each of the plurality of leaflets, the arc length of the free edge is at least 2.5 times greater than the nominal radius of the prosthetic valve.

Example 33. The prosthetic valve of any example herein, particularly example 31, wherein, for each of the plurality of leaflets, the arc length of the free edge is at least 3 times greater than the nominal radius of the prosthetic valve.

Example 34. The prosthetic valve of any example herein, particularly any one of examples 31 to 33, wherein the nominal height of the prosthetic valve is less than twice the nominal radius of the prosthetic valve.

Example 35. A prosthetic valve, comprising:
a frame having an inflow end and an outflow end, wherein the frame is movable between a radially compressed and a radially expanded state; and
a plurality of leaflets secured to the frame, each leaflet having: a leaflet first face and a leaflet second face opposing the leaflet first face, a free edge defining a free edge midpoint, and an opposing cusp edge, the cusp edge defining a cusp edge midpoint and first and second leaflet body sidelines, each leaflet body sideline extending from the cusp edge midpoint to the free edge,
wherein the plurality of leaflets are secured to each other at a plurality of commissure assemblies, each of the plurality of commissure assemblies extending from a commissure assembly outflow end to a commissure assembly inflow end,
wherein the plurality of commissure assemblies are secured to the frame, and
wherein a center of coaptation of the plurality of leaflets is located between the commissure assembly inflow ends and the inflow end of the frame.

Example 36. The prosthetic valve of any example herein, particularly example 35, wherein a commissure outflow plane is defined by the plurality of commissure assembly outflow ends,
wherein a commissure inflow plane is defined by the plurality of commissure assembly inflow ends,
wherein a cusp midpoint plane is defined by the plurality of cusp edge midpoints, and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 37. The prosthetic valve of any example herein, particularly example 35, wherein a cusp midpoint plane is defined by the plurality of cusp edge midpoints, and

wherein a portion of each of the plurality of leaflets is positioned between the cusp midpoint plane and the inflow end of the frame.

Example 38. The prosthetic valve of any example herein, particularly example 37, wherein a commissure outflow plane is defined by the plurality of commissure assembly outflow ends,
wherein a commissure inflow plane is defined by the plurality of commissure assembly inflow ends, and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 39. The prosthetic valve of any example herein, particularly any one of examples 35 to 38, wherein the prosthetic valve has a nominal radius and a nominal height, and wherein, for each of the plurality of leaflets, an arc length of the free edge is at least 2.2 times greater than the nominal radius of the prosthetic valve.

Example 40. The prosthetic valve of any example herein, particularly example 39, wherein, for each of the plurality of leaflets, the arc length of the free edge is at least 2.5 times greater than the nominal radius of the prosthetic valve.

Example 41. The prosthetic valve of any example herein, particularly example 39, wherein, for each of the plurality of leaflets, the arc length of the free edge is at least 3 times greater than the nominal radius of the prosthetic valve.

Example 42. The prosthetic valve of any example herein, particularly any one of examples 39 to 41, wherein the nominal height of the prosthetic valve is less than twice the nominal radius of the prosthetic valve.

Example 43. The prosthetic valve of any example herein, particularly any one of examples 35 to 42, wherein each of the plurality of leaflets is not flattenable.

Example 44. The prosthetic valve of any example herein, particularly any one of examples 35 to 43, wherein, for each of the plurality of leaflets, the leaflet first face and the leaflet second face are non-developable surfaces.

Example 45. The prosthetic valve of any example herein, particularly any one of examples 35 to 44, wherein, for each of the plurality of leaflets, the leaflet first face is concave.

Example 46. The prosthetic valve of any example herein, particularly example 45, wherein, for each of the plurality of leaflets, the concavity of the first face is three-dimensional.

Example 47. The prosthetic valve of any example herein, particularly any one of examples 35 to 46, wherein, for each of the plurality of leaflets, the leaflet second face is convex.

Example 48. The prosthetic valve of any example herein, particularly example 47, wherein, for each of the plurality of leaflets, the convexity of the second face is three-dimensional.

Example 49. The prosthetic valve of any example herein, particularly any one of examples 35 to 48, wherein the plurality of leaflets comprises three leaflets.

Example 50. The prosthetic valve of any example herein, particularly any one of examples 35 to 49, wherein, for each of the plurality of leaflets, the respective leaflet is curved from the first leaflet body sideline to the second leaflet body sideline.

Example 51. The prosthetic valve of any example herein, particularly example 50, wherein the curve of each of the plurality of leaflets from the respective first leaflet body sideline to the respective second leaflet body sideline forms a predetermined section of an ellipsoid.

Example 52. The prosthetic valve of any example herein, particularly any one of examples 50 or 51, wherein each of the first leaflet body sideline and the second leaflet body sideline is curved.

Example 53. The prosthetic valve of any example herein, particularly any one of examples 35 to 52, wherein each of the plurality of leaflets is curved from the free edge to the cusp edge midpoint.

Example 54. The prosthetic valve of any example herein, particularly example 53, wherein an arc length of any curve extending from the free edge to the cusp edge midpoint exhibits a positive correlation with a distance between the respective curve and the closer one of the first leaflet body sideline or the second leaflet body sideline.

Example 55. The prosthetic valve of any example herein, particularly any one of examples 53 or 54, wherein an arc length of a curve extending from the free edge midpoint to the cusp edge midpoint is greater than any other curve extending from the first edge to the cusp edge midpoint.

Example 56. The prosthetic valve of any example herein, particularly any one of examples 35 to 55, wherein the frame defines an enclosure, at least a portion of each of the plurality of leaflets situated within the enclosure.

Example 57. The prosthetic valve of any example herein, particularly example 56, wherein, responsive to flow through the enclosure, a bulge is formed in each of the plurality of leaflets, the bulge extending between the free edge midpoint and the cusp edge midpoint.

Example 58. The prosthetic valve of any example herein, particularly example 57, wherein, for each of the plurality of leaflets, the bulge is formed in the leaflet second face.

Example 59. The prosthetic valve of any example herein, particularly example 58, wherein, for each of the plurality of leaflets, the bulge forms a corresponding indent in the leaflet first face.

Example 60. The prosthetic valve of any example herein, particularly any one of examples 58 or 59, wherein, for each of the plurality of leaflets, the bulge exhibits a pair of opposing bulge sides, each bulge side defining a respective concave curve in the leaflet second face.

Example 61. The prosthetic valve of any example herein, particularly any one of examples 58 or 59, wherein, for each of the plurality of leaflets, the bulge exhibits a bulge first side and an opposite bulge second side, a distance between the bulge first side and the first leaflet body sideline being essentially equal to a distance between the bulge second side the second leaflet body sideline.

Example 62. The prosthetic valve of any example herein, particularly any one of examples 57 to 61, wherein, for each of the plurality of leaflets, the bulge is formed in a central portion of the leaflet.

Example 63. The prosthetic valve of any example herein, particularly any one of examples 57 to 62, wherein, for each of the plurality of leaflets, the bulge extends from a bulge outflow end to a bulge inflow end, a distance between the bulge outflow end and the free edge being greater than a distance between the bulge inflow end and the cusp edge midpoint.

Example 64. The prosthetic valve of any example herein, particularly any one of examples 57 to 62, wherein, for each of the plurality of leaflets, the bulge extends from a bulge outflow end to a bulge inflow end, the bulge inflow end reaching the cusp edge midpoint.

Example 65. The prosthetic valve of any example herein, particularly any one of examples 63 or 64, wherein, for each of the plurality of leaflets, a width of the bulge decreases from the bulge outflow end to the bulge inflow end.

Example 66. The prosthetic valve of any example herein, particularly any one of examples 57 to 65, wherein, for each of the plurality of leaflets, a width of the bulge is 1/9 to ⅓ of a maximal distance between the leaflet body sidelines.

Example 67. The prosthetic valve of any example herein, particularly any one of examples 57 to 66, wherein, for each of the plurality of leaflets, a height to width ratio of the bulge is at least 1:1 to 4:1.

Example 68. The prosthetic valve of any example herein, particularly any one of examples 57 to 67, wherein, for each of the plurality of leaflets, a surface of the bulge is essentially smooth.

Example 69. A prosthetic valve, comprising:
a frame having an inflow end and an outflow end, wherein the frame is movable between a radially compressed and a radially expanded state; and
a plurality of leaflets secured to the frame, each leaflet having: a leaflet first face and a leaflet second face opposing the leaflet first face, a free edge defining a free edge midpoint, and an opposing cusp edge, the cusp edge defining a cusp edge midpoint and first and second leaflet body sidelines, each leaflet body sideline extending from the cusp edge midpoint to the free edge,
wherein, for each of the plurality of leaflets, the respective leaflet is curved from the first leaflet body sideline to the second leaflet body sideline.

Example 70. The prosthetic valve of any example herein, particularly example 69, wherein the curve of each of the plurality of leaflets from the respective first leaflet body sideline to the respective second leaflet body sideline forms a predetermined section of an ellipsoid.

Example 71. The prosthetic valve of any example herein, particularly any one of examples 69 or 70, wherein each of the first leaflet body sideline and the second leaflet body sideline is curved.

Example 72. The prosthetic valve of any example herein, particularly any one of examples 69 to 71, wherein each of the plurality of leaflets is curved from the free edge to the cusp edge midpoint.

Example 73. The prosthetic valve of any example herein, particularly example 72, wherein an arc length of any curve extending from the free edge to the cusp edge midpoint exhibits a positive correlation with a distance between the respective curve and the closer one of the first leaflet body sideline or the second leaflet body sideline.

Example 74. The prosthetic valve of any example herein, particularly any one of examples 72 or 73, wherein an arc length of a curve extending from the free edge midpoint to the cusp edge midpoint is greater than any other curve extending from the first edge to the cusp edge midpoint.

Example 75. The prosthetic valve of any example herein, particularly any one of examples 69 to 74, wherein, for each of the plurality of leaflets, the leaflet first face is concave.

Example 76. The prosthetic valve of any example herein, particularly example 75, wherein, for each of the plurality of leaflets, the concavity of the first face is three-dimensional.

Example 77. The prosthetic valve of any example herein, particularly any one of examples 69 to 76, wherein, for each of the plurality of leaflets, the leaflet second face is convex.

Example 78. The prosthetic valve of any example herein, particularly example 77, wherein, for each of the plurality of leaflets, the convexity of the second face is three-dimensional.

Example 79. The prosthetic valve of any example herein, particularly any one of examples 69 to 78, wherein the plurality of leaflets comprises three leaflets.

Example 80. The prosthetic valve of any example herein, particularly any one of examples 69 to 79, wherein each of the plurality of leaflets is not flattenable.

Example 81. The prosthetic valve of any example herein, particularly any one of examples 69 to 80, wherein, for each of the plurality of leaflets, the leaflet first face and the leaflet second face are non-developable surfaces.

Example 82. The prosthetic valve of any example herein, particularly any one of examples 69 to 81, wherein the frame defines an enclosure, at least a portion of each of the plurality of leaflets situated within the enclosure.

Example 83. The prosthetic valve of any example herein, particularly example 82, wherein, responsive to flow through the enclosure, a bulge is formed in each of the plurality of leaflets, the bulge extending between the free edge midpoint and the cusp edge midpoint.

Example 84. The prosthetic valve of any example herein, particularly example 83, wherein, for each of the plurality of leaflets, the bulge is formed in the leaflet second face.

Example 85. The prosthetic valve of any example herein, particularly example 84, wherein, for each of the plurality of leaflets, the bulge forms a corresponding indent in the leaflet first face.

Example 86. The prosthetic valve of any example herein, particularly any one of examples 84 or 85, wherein, for each of the plurality of leaflets, the bulge exhibits a pair of opposing bulge sides, each bulge side defining a respective concave curve in the leaflet second face.

Example 87. The prosthetic valve of any example herein, particularly any one of examples 84 or 85, wherein, for each of the plurality of leaflets, the bulge exhibits a bulge first side and an opposite bulge second side, a distance between the bulge first side and the first leaflet body sideline being essentially equal to a distance between the bulge second side the second leaflet body sideline.

Example 88. The prosthetic valve of any example herein, particularly any one of examples 83 to 87, wherein, for each of the plurality of leaflets, the bulge is formed in a central portion of the leaflet.

Example 89. The prosthetic valve of any example herein, particularly any one of examples 83 to 88, wherein, for each of the plurality of leaflets, the bulge extends from a bulge outflow end to a bulge inflow end, a distance between the bulge outflow end and the free edge being greater than a distance between the bulge inflow end and the cusp edge midpoint.

Example 90. The prosthetic valve of any example herein, particularly any one of examples 83 to 88, wherein, for each of the plurality of leaflets, the bulge extends from a bulge outflow end to a bulge inflow end, the bulge inflow end reaching the cusp edge midpoint.

Example 91. The prosthetic valve of any example herein, particularly any one of examples 89 or 90, wherein, for each of the plurality of leaflets, a width of the bulge decreases from the bulge outflow end to the bulge inflow end.

Example 92. The prosthetic valve of any example herein, particularly any one of examples 83 to 91, wherein, for each of the plurality of leaflets, a width of the bulge is 1/9 to ⅓ of a maximal distance between the leaflet body sidelines.

Example 93. The prosthetic valve of any example herein, particularly any one of examples 83 to 92, wherein, for each of the plurality of leaflets, a height to width ratio of the bulge is at least 1:1 to 4:1.

Example 94. The prosthetic valve of any example herein, particularly any one of examples 83 to 93, wherein, for each of the plurality of leaflets, a surface of the bulge is essentially smooth.

Example 95. The prosthetic valve of any example herein, particularly any one of examples 69 to 94, wherein the plurality of leaflets are secured to each other at a plurality of commissure assemblies, each of the plurality of commissure assemblies extending from a commissure assembly outflow end to a commissure assembly inflow end,
wherein the plurality of commissure assemblies are secured to the frame, and
wherein a center of coaptation of the plurality of leaflets is located between the commissure assembly inflow ends and the inflow end of the frame.

Example 96. The prosthetic valve of any example herein, particularly example 95, wherein a commissure outflow plane is defined by the plurality of commissure assembly outflow ends,
wherein a commissure inflow plane is defined by the plurality of commissure assembly inflow ends,
wherein a cusp midpoint plane is defined by the plurality of cusp edge midpoints,
and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 97. The prosthetic valve of any example herein, particularly example 95, wherein a cusp midpoint plane is defined by the plurality of cusp edge midpoints, and wherein a portion of each of the plurality of leaflets is positioned between the cusp midpoint plane and the inflow end of the frame.

Example 98. The prosthetic valve of any example herein, particularly example 97, wherein a commissure outflow plane is defined by the plurality of commissure assembly outflow ends,
wherein a commissure inflow plane is defined by the plurality of commissure assembly inflow ends, and
wherein the center of coaptation is located between:
   the inflow end of the frame; and
   the commissure inflow plane, the distance of the center of coaptation to the commissure outflow plane being greater than a third of a distance between the commissure outflow plane and the cusp midpoint plane.

Example 99. The prosthetic valve of any example herein, particularly any one of examples 69 to 98, wherein the prosthetic valve has a nominal radius and a nominal height, and wherein, for each of the plurality of leaflets, an arc length of the free edge is at least 2.2 times greater than the nominal radius of the prosthetic valve.

Example 100. The prosthetic valve of any example herein, particularly example 99, wherein, for each of the plurality of leaflets, the arc length of the free edge is at least 2.5 times greater than the nominal radius of the prosthetic valve.

Example 101. The prosthetic valve of any example herein, particularly example 99, wherein, for each of the plurality of leaflets, the arc length of the free edge is at least 3 times greater than the nominal radius of the prosthetic valve.

Example 102. The prosthetic valve of any example herein, particularly any one of examples 99 to 101, wherein the nominal height of the prosthetic valve is less than twice the nominal radius of the prosthetic valve.

Example 103. A prosthetic valve, comprising:
a frame movable between a radially compressed and a radially expanded state, the frame comprising a plurality of commissure windows, each commissure window comprising two sidewalls and defining a commissure window inner surface and a commissure window outer surface; and
a plurality of leaflets, each leaflet comprising two opposite tabs, wherein the tabs of adjacent leaflets are coupled to each other to form commissure assemblies;
wherein each commissure assembly is coupled to a corresponding commissure window; and
wherein each commissure assembly comprises a separator having a separator thickness and defining a separator inner end, the separator disposed between portions of the tabs extending through the commissure window.

Example 104. The prosthetic valve of any example herein, particularly example 103, wherein the separator thickness is in the range of 100μ and 1 mm.

Example 105. The prosthetic valve of any example herein, particularly example 104, wherein the separator thickness is in the range of 200μ and 500 μ.

Example 106. The prosthetic valve of any example herein, particularly example 104, wherein the separator thickness is in the range of 200μ and 400 μ.

Example 107. The prosthetic valve of any example herein, particularly example 105, wherein the separator thickness is in the range of 250μ and 350 μ.

Example 108. The prosthetic valve of any example herein, particularly example 103, wherein the separator thickness is at least 300 μ.

Example 109. The prosthetic valve of any example herein, particularly any one of examples 103 to 108, wherein a radial distance between the separator inner end and the commissure window inner surface is not greater than the separator thickness.

Example 110. The prosthetic valve of any example herein, particularly any one of examples 103 to 109, wherein the separator is made of thermoplastic polyurethane.

Example 111. The prosthetic valve of any example herein, particularly any one of examples 103 to 109, wherein the separator is coated by thermoplastic polyurethane.

Example 112. The prosthetic valve of any example herein, particularly any one of examples 103 to 111, wherein both tabs of each commissure assembly are coupled to the separator by at least one suture.

Example 113. The prosthetic valve of any example herein, particularly any one of examples 103 to 112, wherein each commissure assembly comprises a coupling member disposed around both of the tabs, and wherein the separator is formed as an integral part of the coupling member which is folded over itself between both of the tabs.

Example 113. The prosthetic valve of any example herein, particularly any one of examples 103 to 109, wherein the separator is a T-shaped separator comprising a radial extension disposed between the portions of the tabs extending through the commissure window, and a lateral head perpendicular to the radial extension, wherein the radial extension has the separator thickness and defines the separator inner end.

Example 114. The prosthetic valve of any example herein, particularly example 113, wherein the T-shaped separator is made of a rigid material.

Example 115. The prosthetic valve of any example herein, particularly any one of examples 113 or 114, wherein the lateral portions is disposed radially away from the commissure window outer surface.

Example 116. The prosthetic valve of any example herein, particularly any one of examples 113 to 115, wherein the tabs of each commissure assembly are wrapped over the lateral portion of the T-shaped separator.

Example 117. The prosthetic valve of any example herein, particularly any one of examples 113 to 116, wherein each tab comprises:
a first tab portion disposed between the radial extension and a corresponding one of the sidewalls, the first tab portion extending in a radial direction through the commissure window and terminating at a first fold;
a second tab portion disposed between the lateral head and the corresponding sidewall, the second tab portion extending in a lateral direction from the first fold to a second fold;
a third tab portion extending in a radial direction from the second fold to a third fold; and a fourth tab portions extending in a lateral direction from the third fold to a tab edge;
wherein the fourth tab portion of a first tab of the tabs of a commissure assembly extends over and in contact with the lateral head, and fourth tab portion of a second tab of the tabs of the same commissure assembly extends at least partially over the fourth tab portion of the first tab.

Example 118. The prosthetic valve of any example herein, particularly example 117, wherein each commissure assembly further comprises:
two first sutures, each forming an overcast stitch pattern along a corresponding fourth tab portion, each first suture comprising:
   an in-and-out stitch line distanced from the third fold of the corresponding tab; and
   a series of bights extending from the in-and-out stitch line and looped over the corresponding tab edge; and
a second suture passed in a zig-zag pattern between stitches of the in-and-out stitch line of the first tab and bights of the second tab, the second suture comprising:
   a series of first wraps, each wrapped around a stitch of the in-and-out stitch line extending along the first tab; and
   a series of second wraps, each wrapped around a bight of the first suture stitched through the second tab.

Example 119. The prosthetic valve of any example herein, particularly example 118, wherein each commissure assembly further comprises:
two third sutures, each extending between one of the in-and-out stitch lines and around a corresponding one of the sidewalls, each third suture comprising:
   a series of sidewall loops, each looped around the corresponding sidewall; and
   a series of intermediary wraps extending between adjacent sidewall loops, each intermediary wrap passing between the fourth tab portion and one stitch on one side of a penetration point of the in-and-out stitch line, and between the fourth tab portion and another stitch on the other side of the penetration point.

Example 120. The prosthetic valve of any example herein, particularly any one of examples 113 to 119, wherein the T-shaped separator is formed from a plate bent over itself to form the radial extension, wherein the plate has a thickness not greater than half the separator thickness.

Example 121. The prosthetic valve of any example herein, particularly example 120, wherein both portions of the plate forming the radial extension are naturally biased away from each other.

Example 122. A prosthetic valve, comprising:
a frame having an inflow end and an outflow end, wherein the frame is movable between a radially compressed and a radially expanded state; and
a plurality of leaflets secured to the frame, each leaflet having: a leaflet first face and a leaflet second face opposing the leaflet first face, a free edge and an opposing cusp edge, the cusp edge defining first and second leaflet body sidelines,
each leaflet body sideline extending from a midpoint of the cusp edge midpoint to the free edge,
wherein each of the plurality of leaflets comprises a plurality of lateral folds extending between the leaflet body sidelines.

Example 123. The prosthetic valve of any example herein, particularly example 122, wherein the plurality of lateral folds of each leaflet comprises at least three lateral folds.

Example 124. The prosthetic valve of any example herein, particularly example 122, wherein the plurality of lateral folds of each leaflet comprises at least five lateral folds.

Example 125. The prosthetic valve of any example herein, particularly example 122, wherein the plurality of lateral folds of each leaflet comprises at least eight lateral folds.

Example 126. The prosthetic valve of any example herein, particularly example 122, wherein the plurality of lateral folds of each leaflet comprises at least ten lateral folds.

Example 127. The prosthetic valve of any example herein, particularly any one of examples 122 to 126, wherein the leaflet has a leaflet thickness defined between the leaflet first face and the leaflet second face, wherein the plurality of lateral folds define a plurality of inner peaks along the leaflet second face and a corresponding plurality of outer valleys along the leaflet first face.

Example 128. The prosthetic valve of any example herein, particularly example 127, wherein each lateral fold defines a maximal fold depth that is greater than the leaflet thickness.

Example 129. The prosthetic valve of any example herein, particularly example 128, wherein the maximal fold depth is at least three times as great as the leaflet thickness.

Example 130. The prosthetic valve of any example herein, particularly example 128, wherein the maximal fold depth is at least five times as great as the leaflet thickness.

Example 131. The prosthetic valve of any example herein, particularly example 128, wherein the maximal fold depth is at least eight times as great as the leaflet thickness.

Example 132. The prosthetic valve of any example herein, particularly example 128, wherein the maximal fold depth is at least ten times as great as the leaflet thickness.

Example 133. The prosthetic valve of any example herein, particularly any one of examples 127 to 132, wherein each two adjacent lateral folds define a fold-spacing there-between, which is greater than the leaflet thickness.

Example 134. The prosthetic valve of any example herein, particularly example 133, wherein the minimal fold spacing is at least three times as great as the leaflet thickness.

Example 135. The prosthetic valve of any example herein, particularly example 133, wherein the minimal fold spacing is at least five times as great as the leaflet thickness.

Example 136. The prosthetic valve of any example herein, particularly example 133, wherein the minimal fold spacing is at least eight times as great as the leaflet thickness.

Example 137. The prosthetic valve of any example herein, particularly example 133, wherein the minimal fold spacing is at least ten times as great as the leaflet thickness.

Example 138. The prosthetic valve of any example herein, particularly any one of examples 133 to 137, wherein the maximal fold spacing is at least three times as great as the leaflet thickness.

Example 139. The prosthetic valve of any example herein, particularly any one of examples 133 to 137, wherein the maximal fold spacing is at least five times as great as the leaflet thickness.

Example 140. The prosthetic valve of any example herein, particularly any one of examples 133 to 137, wherein the maximal fold spacing is at least eight times as great as the leaflet thickness.

Example 141. The prosthetic valve of any example herein, particularly any one of examples 133 to 137, wherein the maximal fold spacing is at least ten times as great as the leaflet thickness.

Example 142. The prosthetic valve of any example herein, particularly any one of examples 122 to 126, wherein the leaflet first face is devoid of peaks and valleys.

Example 143. The prosthetic valve of any example herein, particularly example 144, wherein the leaflet comprises an inner layer comprising the lateral folds and having an inner layer thickness between the leaflet second face and an opposite inner layer first face, and an outer layer defined extending between the leaflet first face and an opposite outer layer second face attached to the inner layer first face.

Example 144. The prosthetic valve of any example herein, particularly example 143, wherein the outer layer comprises an elastic compressible material.

Example 145. The prosthetic valve of any example herein, particularly example 143 or 144, wherein the lateral folds define a plurality of inner peaks along the leaflet second face and a corresponding plurality of inner layer outer valleys along the inner layer first face, and wherein the outer layer second face comprises a plurality of outer layer peaks extending into the inner layer outer valleys, and a plurality of outer layer valleys disposed between the outer layer peaks.

Example 146. The prosthetic valve of any example herein, particularly example 145, wherein the inner layer has an inner layer thickness defined between the leaflet second face and the inner layer first face, wherein the outer layer has a minimal outer layer thickness defined between the leaflet first face and the outer layer valleys, and wherein the outer layer thickness is greater than the minimal inner layer thickness.

Example 147. The prosthetic valve of any example herein, particularly example 146, wherein the outer layer thickness is at least three times greater than the minimal outer layer thickness.

Example 148. The prosthetic valve of any example herein, particularly example 146, wherein the outer layer thickness is at least five times greater than the minimal outer layer thickness.

Example 149. The prosthetic valve of any example herein, particularly example 146, wherein the outer layer thickness is at least ten times greater than the minimal outer layer thickness.

Example 150. The prosthetic valve of any example herein, particularly any one of examples 146 to 149, wherein each lateral fold defines a maximal fold depth that is greater than the inner layer thickness.

Example 151. The prosthetic valve of any example herein, particularly example 150, wherein the maximal fold depth is at least three times as great as the inner layer thickness.

Example 152. The prosthetic valve of any example herein, particularly example 150, wherein the maximal fold depth is at least five times as great as the inner layer thickness.

Example 153. The prosthetic valve of any example herein, particularly example 150, wherein the maximal fold depth is at least eight times as great as the inner layer thickness.

Example 154. The prosthetic valve of any example herein, particularly example 150, wherein the maximal fold depth is at least ten times as great as the inner layer thickness.

Example 155. The prosthetic valve of any example herein, particularly any one of examples 146 to 154, wherein each two adjacent lateral folds define a fold-spacing there-between, which is greater than the inner layer thickness.

Example 156. The prosthetic valve of any example herein, particularly example 155, wherein the minimal fold spacing is at least three times as great as the inner layer thickness.

Example 157. The prosthetic valve of any example herein, particularly example 155, wherein the minimal fold spacing is at least five times as great as the inner layer thickness.

Example 158. The prosthetic valve of any example herein, particularly example 155, wherein the minimal fold spacing is at least eight times as great as the inner layer thickness.

Example 159. The prosthetic valve of any example herein, particularly example 155, wherein the minimal fold spacing is at least ten times as great as the inner layer thickness.

Example 160. The prosthetic valve of any example herein, particularly any one of examples 155 to 159, wherein the maximal fold spacing is at least three times as great as the inner layer thickness.

Example 161. The prosthetic valve of any example herein, particularly any one of examples 155 to 159, wherein the maximal fold spacing is at least five times as great as the inner layer thickness.

Example 162. The prosthetic valve of any example herein, particularly any one of examples 155 to 159, wherein the maximal fold spacing is at least eight times as great as the inner layer thickness.

Example 163. The prosthetic valve of any example herein, particularly any one of examples 155 to 159, wherein the maximal fold spacing is at least ten times as great as the inner layer thickness.

Example 164. The prosthetic valve of any example herein, particularly any one of examples 122 to 163, wherein the leaflet body is gathered along both leaflet body sidelines.

Example 165. The prosthetic valve of any example herein, particularly any one of examples 122 to 164, wherein the plurality of leaflets comprises three leaflets.

Example 166. The prosthetic valve of any example herein, particularly any one of examples 122 to 165, wherein the leaflet has a leaflet body height, and wherein the leaflet is configured to transition between a free state and a stretched state such that the leaflet body height in the stretched state is greater than the leaflet body height in the free state.

It is appreciated that certain features of the invention, which are, for clarity, described in the context of separate examples, may also be provided in combination in a single example. Conversely, various features of the invention, which are, for brevity, described in the context of a single example, may also be provided separately or in any suitable sub-combination or as suitable in any other described example of the invention. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve, comprising:
a frame having an inflow end and an outflow end, wherein the frame is movable between a radially compressed and a radially expanded state; and
a plurality of leaflets secured to the frame, each leaflet having: a leaflet first face and a leaflet second face opposing the leaflet first face, a free edge defining a free edge midpoint, and an opposing cusp edge, the cusp edge defining a cusp edge midpoint and first and second leaflet body sidelines, each leaflet body sideline extending from the cusp edge midpoint to the free edge,
wherein the frame defines an enclosure, at least a portion of each of the plurality of leaflets situated within the enclosure, and
wherein, responsive to flow through the enclosure, a bulge is formed in each of the plurality of leaflets, the bulge extending between the free edge midpoint and the cusp edge midpoint.

2. The prosthetic valve of claim 1, wherein, for each of the plurality of leaflets, the bulge is formed in the leaflet second face.

3. The prosthetic valve of claim 2, wherein, for each of the plurality of leaflets, the bulge forms a corresponding indent in the leaflet first face.

4. The prosthetic valve of claim 1, wherein, for each of the plurality of leaflets, the bulge extends from a bulge outflow end to a bulge inflow end, the bulge inflow end reaching the cusp edge midpoint.

5. The prosthetic valve of claim 1, wherein, for each of the plurality of leaflets, a width of the bulge decreases from the bulge outflow end to the bulge inflow end.

6. The prosthetic valve of claim 1, wherein, for each of the plurality of leaflets, a width of the bulge is 1/9 to ⅓ of a maximal distance between the leaflet body sidelines.

7. The prosthetic valve of claim 1, wherein, for each of the plurality of leaflets, a height to width ratio of the bulge is at least 1:1 to 4:1.

8. The prosthetic valve of claim 1, wherein each of the plurality of leaflets is non-flattenable.

9. The prosthetic valve of claim 8, wherein, for each of the plurality of leaflets, the leaflet first face and the leaflet second face are non-developable surfaces.

10. The prosthetic valve of claim 8, wherein, for each of the plurality of leaflets, the respective leaflet is curved from the first leaflet body sideline to the second leaflet body sideline.

11. The prosthetic valve of claim 10, wherein the curve of each of the plurality of leaflets from the respective first leaflet body sideline to the respective second leaflet body sideline forms a predetermined section of an ellipsoid.

12. The prosthetic valve of claim 8, wherein each of the plurality of leaflets is curved from the free edge to the cusp edge midpoint.

13. The prosthetic valve of claim 12, wherein an arc length of any curve extending from the free edge to the cusp edge midpoint exhibits a positive correlation with a distance between the respective curve and the closer one of the first leaflet body sideline or the second leaflet body sideline.

14. The prosthetic valve of claim 12, wherein an arc length of a curve extending from the free edge midpoint to the cusp edge midpoint is greater than any other curve extending from the first edge to the cusp edge midpoint.

15. The prosthetic valve of claim 1, wherein the plurality of leaflets are secured to each other at a plurality of commissure assemblies, each of the plurality of commissure assemblies extending from a commissure assembly outflow end to a commissure assembly inflow end,
wherein the plurality of commissure assemblies are secured to the frame, and
wherein a center of coaptation of the plurality of leaflets is located between the commissure assembly inflow ends and the inflow end of the frame.

16. The prosthetic valve of claim 15, wherein a commissure outflow plane is defined by the plurality of commissure assembly outflow ends,
wherein a commissure inflow plane is defined by the plurality of commissure assembly inflow ends,
wherein a cusp midpoint plane is defined by the plurality of cusp edge midpoints, and
wherein the center of coaptation is located between:
the inflow end of the frame; and
the commissure inflow plane, the distance of the center of coaptation to the commissure outflow plane being greater than a third of a distance between the commissure outflow plane and the cusp midpoint plane.

17. The prosthetic valve of claim 16, wherein a cusp midpoint plane is defined by the plurality of cusp edge midpoints, and
wherein a portion of each of the plurality of leaflets is positioned between the cusp midpoint plane and the inflow end of the frame.

18. The prosthetic valve of claim 17, wherein a commissure outflow plane is defined by the plurality of commissure assembly outflow ends,
wherein a commissure inflow plane is defined by the plurality of commissure assembly inflow ends, and
wherein the center of coaptation is located between:
the inflow end of the frame; and
the commissure inflow plane, the distance of the center of coaptation to the commissure outflow plane being greater than a third of a distance between the commissure outflow plane and the cusp midpoint plane.

19. The prosthetic valve of claim 1, wherein the prosthetic valve has a nominal radius and a nominal height, and wherein, for each of the plurality of leaflets, an arc length of the free edge is at least 2.2 times greater than the nominal radius of the prosthetic valve.

20. The prosthetic valve of claim 19, wherein the nominal height of the prosthetic valve is less than twice the nominal radius of the prosthetic valve.
